(19) United States
(12) Patent Application Publication
(10) Pub. No.: US 2006/0079675 A1
(43) Pub. Date: Apr. 13, 2006

(54) Compositions and use of inflammation-20 (Inf-20) gene

(76) Inventors: Youhai H. Chen, Newtown Square, PA (US); Honghong Sun, Swarthmore, PA (US); Shunyou Gong, Philadelphia, PA (US)

Correspondence Address: PEARL COHEN ZEDEK, LLP, 10 ROCKEFELLER PLAZA, SUITE 1001, NEW YORK, NY, 10020, US

(21) Appl. No.: 11/122,256

(22) Filed: May 5, 2005

Related U.S. Application Data
(60) Provisional application No. 60/568,561, filed on May 6, 2004.

(51) Int. Cl. C12Q 1/68; C07H 21/04; C12N 9/99

(52) U.S. Cl. 536/23.1; 435/6; 435/69.2; 435/184; 435/320.1

(57) ABSTRACT

This invention relates to Inf-20, a novel apoptosis regulator, that in one embodiment is induced by TNF. The gene, designated inflammation-20 (Inf-20), encodes a polypeptide of 184 amino acids. In one embodiment Inf-20 comprises a conserved death effector domain (DED) that is capable of interacting with caspase-8, but not with Fas-associated death domain (FADD) protein.

### CROSS REFERENCE TO RELATED APPLICATIONS

This application claims priority of provisional patent application U.S. Ser. No. 60/568,561, filed May 6, 2004, the contents of which are hereby incorporated in their entirety.

### GOVERNMENT SUPPORT

This invention was made with support from the National Institutes of Health (grant numbers AI50059, AI055934, AI55934); therefore, the government has certain rights in the invention.

### FIELD OF INVENTION

This invention relates to Inf-20, a novel apoptosis regulator, that in one embodiment is induced by TNF. The gene, designated inflammation-20 (Inf-20), encodes a polypeptide of 184 amino acids. In one embodiment Inf-20 comprises a conserved death effector domain (DED) that is capable of interacting with caspase-8, but not with Fas-associated death domain (FADD) protein.

### BACKGROUND OF THE INVENTION

Multiple sclerosis (MS) is a chronic inflammatory and demyelinating disease of the central nervous system. There is increasing evidence indicating MS is associated with autoimmune inflammation involving activation and aberrant trafficking of T cells and other inflammatory cells which produce an array of inflammatory molecules such as. cytokines, chemokines, their receptors and molecules related to T cell adhesion, trafficking and apoptosis. The production of these molecules not only characteristically reflects the in vivo activity of inflammatory cells but also has clinical relevance to disease activity in MS. According to current hypotheses, activated autoreactive CD4+ T helper cells (Th1 cells) which preferentially secrete interferon-gamma (INF-γ) and tumor necrosis factors alpha/beta (TNF-alpha/beta.), induce inflammation and demyelination in MS.

Therapeutic advances in multiple sclerosis (MS) have been slow to emerge, partly because of incomplete understanding of the pathogenesis of the disorder. For empirically based treatment the major obstacles to progress include the highly variable course of MS, the long-term nature of the most important outcome measures, and the lack of objective markers of treatment effect, particularly in the short term.

Excessive apoptosis is associated with a wide range of human diseases, and the importance of caspases in the progression of many of these disorders has been demonstrated with both small molecule and peptide-based regulators as well as by genetic approaches. Caspase regulators have been suggested to offer therapeutic benefit in numerous acute disorders, such as cardiac and cerebral ischemia/reperfusion injury (e.g. stroke), spinal cord injury, traumatic brain injury, organ damage during transplantation, liver degeneration (as caused, for example, by hepatitis), sepsis, bacterial meningitis and a number of dermatological conditions. There are also a wide range of chronic disorders in which excessive apoptosis is implicated, such as neurodegenerative diseases (e.g. Alzheimer's disease, polyglutamine-repeat disorders such as Huntington's Disease, Down's Syndrome, spinal muscular atrophy, multiple sclerosis, Parkinson's disease), immunodeficiency diseases (e. g. HIV), arthritis, atherosclerosis, diabetes, alopecia, and aging. Caspase regulators could also be used to extend the lifespan of purified blood products to be used for transfusions, or to enhance the lifespan of donated organs before transplantation. Thus, small molecule regulators of either Group I, II, or III caspases (initiator caspases, to which Caspase-8 belongs), are likely to have tremendous therapeutic benefit.

Experimental autoimmune encephalomyelitis (EAE) is an inflammatory disease of the central nervous system (CNS) that has long been used as an animal model for human multiple sclerosis (MS). The active inflammatory process in EAE and MS is confined to the white matter in the central nervous system, not affecting the peripheral nervous tissues. One of the hallmarks of MS/EAE pathology is demyelination and death of oligodendrocytes that produce the myelin sheath. Using in situ TUNEL technique, a method that detects apoptosis sensitively at the single cell level, The acute MS plaques were shown to contain massive numbers of inflammatory and glial cells undergoing apoptosis. The presence of apoptotic cells in the MS plaques was also confirmed using other techniques such as confocal microscopy and electrophoresis of the DNA isolated from MS subjects' brains. Similarly, apoptosis may directly contribute to the pathology of EAE. By combined immunohistochemistry and in situ nick labeling, large numbers of apoptotic cells were detected in the brain and spinal cord of Lewis rats, during the acute phase of EAE Surprisingly, up to 50% of T lymphocytes in EAE lesions showed signs of apoptosis. Recently, using several mouse models of EAE, large numbers of apoptotic cells in the central were detected in the nervous system, both at peak of the disease and during disease recovery.

The mechanisms by which apoptosis is initiated and regulated in MS and EAE are not well understood. In acute MS/EAE plaques, various apoptosis-inducing molecules are present. These include reactive oxygen species (ROS) and cytotoxic enzymes as well as pro-apoptotic members of the tumor necrosis factor (TNF) family.

### SUMMARY OF THE INVENTION

In one embodiment, the invention provides an isolated Inf-20 nucleic acid molecule, encoding an apoptosis inhibition protein, wherein said Inf-20 has a nucleic acid sequence as set forth in SEQ ID NO. 1.

In another embodiment, the invention provides an oligonucleotide of at least 15 nucleotides capable of specifically hybridizing with a sequence of said nucleic acid, which encodes the sequence as set forth in SEQ ID NO. 1.

In one embodiment, the invention provide a nucleic acid having a sequence complementary to the Inf-20 sequence of the isolated nucleic acid molecule, encoding an apoptosis inhibition protein, wherein said Inf-20 has a nucleic acid sequence as set forth in SEQ ID NO. 1.

In another embodiment, the invention provides an antisense molecule capable of specifically hybridizing with the Inf-20 isolated nucleic acid molecule, encoding an apoptosis inhibition protein, wherein said Inf-20 has a nucleic acid sequence as set forth in SEQ ID NO. 1.

In one embodiment, the invention provides a transgenic, nonhuman organism comprising the Inf-20 isolated nucleic acid molecule, encoding an apoptosis inhibition protein, wherein said Inf-20 has a nucleic acid sequence as set forth in SEQ ID NO. 1.

In another embodiment, the invention provides a vector comprising the Inf-20 isolated nucleic acid molecule, encoding an apoptosis inhibition protein, wherein said Inf-20 has a nucleic acid sequence as set forth in SEQ ID NO. 1.

In one embodiment, the invention provides an isolated inf-20 polypeptide selected from: a polypeptide comprising the amino acid sequence as set forth in SEQ ID NO:2, a polypeptide comprising a naturally occurring amino acid sequence at least 72% identical to the amino acid sequence as set forth in SEQ ID NO:2, wherein said polypeptide has TNF-α or FADD induced apoptosis inhibition activity and is able to interact with caspase-8, a biologically active fragment of a polypeptide having an amino acid sequence of SEQ ID NO:2, wherein said fragment has TNF-α or FADD induced apoptosis inhibition activity, or a polypeptide encoded by the nucleic acid sequence as set forth in SEQ ID NO. 1.

In another embodiment, the invention provides a composition, comprising a inf-20 encoded polypeptide, represented by SEQ ID NO. 1 and a pharmaceutically acceptable carrier, excipient, flow agent, processing aid, diluent or a combination thereof.

In one embodiment, the invention provides a method for identifying, or in another embodiment, diagnosing or in another embodiment, predicting a candidate for developing a neurodegenerative disease comprising determining whether inf-20 is overexpressed as compared with a standard.

In another embodiment, the invention provides a method of reducing symptoms of a neurodegenerative disease, or in another embodiment, treating, or in another embodiment preventing or inhibiting a neurodegenerative disease in a subject, comprising administering to said subject the composition comprising an Inf-20 encoded polypeptide, represented by SEQ ID NO. 1 and a pharmaceutically acceptable carrier, excipient, flow agent, processing aid, diluent or a combination thereof, in an amount effective to reduce symptoms of said neurodegenerative disease.

In one embodiment, the invention provides a medium having disposed thereon an oligonucleotide-hybridized cRNA of inf-20.

In another embodiment, the invention provides a method of regulating death receptor induced apoptosis in a subject, comprising administering to said subject an isolated inf-20 polypeptide selected from: a polypeptide comprising the amino acid sequence as set forth in SEQ ID NO:2, a polypeptide comprising a naturally occurring amino acid sequence at least 72% identical to the amino acid sequence as set forth in SEQ ID NO:2, wherein said polypeptide has TNF-α or FADD induced apoptosis inhibition activity and is able to interact with caspase-8, a biologically active fragment of a polypeptide having an amino acid sequence of SEQ ID NO:2, wherein said fragment has TNF-α or FADD induced apoptosis inhibition activity, or a polypeptide encoded by the nucleic acid sequence as set forth in SEQ ID NO. 1, in an amount effective to regulate death receptor induced Apoptosis in said subject.

In one embodiment, the invention provides a method of regulating death receptor induced apoptosis in a cell, comprising contacting said cell with an isolated inf-20 polypeptide selected from: a polypeptide comprising the amino acid sequence as set forth in SEQ ID NO:2, a polypeptide comprising a naturally occurring amino acid sequence at least 72% identical to the amino acid sequence as set forth in SEQ ID NO:2, wherein said polypeptide has TNF-α or FADD induced apoptosis inhibition activity and is able to interact with caspase-8, a biologically active fragment of a polypeptide having an amino acid sequence of SEQ ID NO:2, wherein said fragment has TNF-α or FADD induced apoptosis inhibition activity, or a polypeptide encoded by the nucleic acid sequence as set forth in SEQ ID NO. 1, in an amount effective to regulate death receptor induced apoptosis in said cell.

In another embodiment, the invention provides a method of promoting cell survival, comprising increasing the expression of inf-20, wherein increasing said expression of said inf-20 is by contacting said cell with an inf-20 agonist.

In one embodiment, the invention provides a method of screening a compound for effectiveness as an agonist, or in another embodiment, as an antagonist of a polypeptide selected from: a polypeptide comprising the amino acid sequence as set forth in SEQ ID NO:2, a polypeptide comprising a naturally occurring amino acid sequence at least 72% identical to the amino acid sequence as set forth in SEQ ID NO:2, wherein said polypeptide has TNF-α or FADD induced apoptosis inhibition activity and is able to interact with caspase-8, a biologically active fragment of a polypeptide having an amino acid sequence of SEQ ID NO:2, wherein said fragment has TNF-α or FADD induced apoptosis inhibition activity, or a polypeptide encoded by the nucleic acid sequence as set forth in SEQ ID NO. 1, the method comprising; contacting a sample comprising the polypeptide selected from: a polypeptide comprising the amino acid sequence as set forth in SEQ ID NO:2, a polypeptide comprising a naturally occurring amino acid sequence at least 72% identical to the amino acid sequence as set forth in SEQ ID NO:2, wherein said polypeptide has TNF-α or FADD induced apoptosis inhibition activity and is able to interact with caspase-8, a biologically active fragment of a polypeptide having an amino acid sequence of SEQ ID NO:2, wherein said fragment has TNF-α or FADD induced apoptosis inhibition activity, or a polypeptide encoded by the nucleic acid sequence as set forth in SEQ ID NO. 1 to a compound, and determining if TNF-α or FADD did induce apoptosis inhibition activity, or in another embodiment, did not induce apoptosis inhibition in said sample is increased or decreased in comparison to a control sample lacking said compound.

In one embodiment, the invention provides a method of blocking apoptosis in a cell comprising contacting said cell with the polypeptide selected from: a polypeptide comprising the amino acid sequence as set forth in SEQ ID NO:2, a polypeptide comprising a naturally occurring amino acid sequence at least 72% identical to the amino acid sequence as set forth in SEQ ID NO:2, wherein said polypeptide has TNF-α or FADD induced apoptosis inhibition activity and is able to interact with caspase-8, a biologically active fragment of a polypeptide having an amino acid sequence of SEQ ID NO:2, wherein said fragment has TNF-α or FADD induced apoptosis inhibition activity, or a polypeptide encoded by the nucleic acid sequence as set forth in SEQ ID NO. 1, wherein said polypeptide binds to caspase-8, therefore blocking apoptosis by directly acting on caspases.

In another embodiment, the invention provides a method of inhibiting TNF-α induced apoptosis, or in one embodiment FADD induced apoptosis in a cell, comprising increasing the expression of inf-20 in said cell thereby inhibiting TNF-α or in another embodiment FADD induced apoptosis in said cell.

### BRIEF DESCRIPTION OF THE DRAWINGS

FIG. 1 shows BLAST alignment results for Inf-20 with various EST's

FIG. 2 shows a gel electrophoresis slide, indicating high levels of Inf-20 mRNA in the spinal cord and brain of EAE mice.

FIG. 3 shows the results of pre-incubation of cells with TNF as inducing significant levels of Inf-20 gene expression.

FIG. 4 shows Co-immunoprecipitation analyses of FLAG-tagged Inf-20 and HA-tagged FADD following transfection with corresponding expression plasmids.

FIG. 5 shows Inf-20 expression significantly inhibited FADD induce apoptosis in more than 75% of transfected HeLa cells.

FIG. 6 shows TRAF-2 expression markedly increasing NF-κB activity, contrasted by Inf-20 transfection having little effect on NF-κB activation at low doses, and only slightly increased the NF-κB activity at a very high dose.

FIG. 7 shows Inf-20's capability of enhancing apoptosis of primary T cells and EL4 T cell line.

FIG. 8 shows how Inf-20 inhibits the proliferation of T cells

### DETAILED DESCRIPTION OF THE INVENTION

In one embodiment, Inf-20 as described herein is generally a TNF-induced gene that promotes cell survival.

While apoptosis is a hallmark of pathology in inflammatory diseases, it can be either beneficial or detrimental to the target tissues (such as the nervous tissue in EAE). Apoptosis of inflammatory cells promotes resolution of inflammation and prevents tissue injury. By contrast, apoptosis of resident cells in the inflamed tissue may lead to tissue injury and exacerbate the disease. Interestingly, both inflammatory cells and tissue cells are equipped with similar cell death machinery (e.g., caspases and death receptors) that can be activated by TNF, FasL, reactive oxygen species and cytotoxic enzymes. Effective regulation of the apoptosis apparatus is therefore critical for maintaining the integrity of the organ systems. As described herein Inf-20 is such a molecule, capable of regulating apoptosis during inflammation.

Therefore, in this aspect of the invention and in one embodiment, the invention provides an isolated Inf-20 nucleic acid, encoding an apoptosis inhibition protein, induced by TNF-α or FADD, wherein the inf-20 has a nucleic acid sequence as set forth in SEQ ID NO. 1, including, in another embodiment, variants or mutants thereof having apoptosis inhibition activity, induced by TNF-α or FADD.

In nother embodiment, the term “apoptosis” refers to programmed cell death, which in one embodiment is a principal mechanism by which organisms eliminate unwanted cells. Apoptosis, is a mode of cell death that occurs under normal physiological conditions and the cell is an active participant in its own demise (“cellular suicide”). It is most often found during normal cell turnover and tissue homeostasis, embryogenesis, induction and maintenance of immune tolerance, development of the nervous system and endocrine-dependent tissue atrophy. Cells undergoing apoptosis show characteristic morphological and biochemical features. These features include chromatin aggregation, nuclear and cytoplasmic condensation, partition of cytoplasm and nucleus into membrane bound vesicles (apoptotic bodies) which contain ribosomes, morphologically intact mitochondria and nuclear material. In vivo, these apoptotic bodies are rapidly recognized and phagocytized by either macrophages or adjacent epithelial cells. Due to this efficient mechanism for the removal of apoptotic cells in vivo no inflammatory response is elicited. In vitro, the apoptotic bodies as well as the remaining cell fragments ultimately swell and finally lyse. This terminal phase of in vitro cell death has been termed “secondary necrosis”.

In another embodiment, the deregulation of apoptosis, either excessive apoptosis in one embodiment, or the failure to undergo apoptosis in another embodiment, has been implicated in a number of diseases such as cancer in one embodiment, or acute inflammatory and autoimmune disorders in another embodiment, or ischemic diseases in another embodiment or certain neurodegenerative disorders in another embodiment.

The nucleotide encoding inf-20 includes RNA, cDNA, genomic DNA, synthetic forms, and mixed polymers, both sense and antisense strands, and may be chemically or biochemically modified or may contain non-natural or derivatized nucleotide bases, as will be readily appreciated by those skilled in the art. Such modifications include, for example, labels, methylation, substitution of one or more of the naturally occurring nucleotides with an analog, internucleotide modifications such as uncharged linkages (e.g., methyl phosphonates, phosphotriesters, phosphoamidates, carbamates, etc.), charged linkages (e.g., phosphorothioates, phosphorodithioates, etc.), pendent moieties (e.g., polypeptides), intercalators (e.g., acridine, psoralen, etc.), chelators, alkylators, and modified linkages (e.g., alpha anomeric nucleic acids, etc.). Also included are synthetic molecules that mimic nucleotides in their ability to bind to a designated sequence via hydrogen bonding and other chemical interactions. Such molecules are known in the art and include, for example, those in which peptide linkages substitute for phosphate linkages in the backbone of the molecule. substantially homologous to primary structural sequence but which include, e.g., in vivo or in vitro chemical and biochemical modifications or which incorporate unusual amino acids. The nucleic acid may be modified. Such modifications include, for example, acetylation, carboxylation, phosphorylation, glycosylation, ubiquitination, labeling, e.g., with radionuclides, and various enzymatic modifications, as will be readily appreciated by those well skilled in the art. A variety of methods for labeling polypeptides and of substituents or labels useful for such purposes are well known in the art, and include radioactive isotopes such as 32 P, ligands which bind to labeled antiligands (e.g., antibodies), fluorophores, chemiluminescent agents, enzymes, and antiligands which can serve as specific binding pair members for a labeled ligand. The choice of label depends on the sensitivity required, ease of conjugation with the primer, stability requirements, and available instrumentation. Methods of labeling polypeptides are well known in the art. See, e.g., Sambrook et al., 1989 or Ausubel et al., 1992. Besides substantially full-length Inf-20, the present invention provides for biologically active fragments of the Inf-20 which are known to those skilled in the art

In one embodiment, the term “isolated” or “substantially pure” nucleic acid (e.g., an RNA, DNA or a mixed polymer) is one which is substantially separated from other cellular components which naturally accompany a native sequence or protein, e.g., ribosomes, polymerases, many other human genome sequences and proteins. The term embraces a nucleic acid sequence or protein which has been removed from its naturally occurring environment, and includes recombinant or cloned DNA isolates and chemically synthesized analogs or analogs biologically synthesized by heterologous systems.

In another embodiment, the term “nucleic acid” refers to the phosphate ester polymeric form of ribonucleosides (adenosine, guanosine, uridine or cytidine; “RNA molecules”) or deoxyribonucleosides (deoxyadenosine, deoxyguanosine, deoxythymidine, or deoxycytidine; “DNA molecules”) in either single stranded form, or a double-stranded helix. Double stranded DNA-DNA, DNA-RNA and RNA-RNA helices are possible. The term nucleic acid molecule, and in particular DNA or RNA molecule, refers only to the primary and secondary structure of the molecule, and does not lim it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the nontranscribed strand of DNA (i.e., the strand having a sequence homologous to the mRNA). A “recombinant DNA” is a DNA that has undergone a molecular biological manipulation.

The phrase “nucleic acid encoding” refers to a nucleic acid molecule which directs the expression of a specific protein or peptide. The nucleic acid sequences include both the DNA strand sequence that is transcribed into RNA and the RNA sequence that is translated into protein. The nucleic acid molecule include both the full length nucleic acid sequences as well as non-full length sequences derived from the full length protein. It being further understood that the sequence includes the degenerate codons of the native sequence or sequences which may be introduced to provide codon preference in a specific host cell.

In one embodiment, the term “Recombinant nucleic acid” is a nucleic acid which is not naturally occurring, or which is made by the artificial combination of two otherwise separated segments of sequence. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions.

The following terms are used to describe the sequence relationships between two or more nucleic acid molecules or polynucleotides: “reference sequence”, “comparison window”, “sequence identity”, “percentage of sequence identity”, and “substantial identity”. In one embodiment, the term “reference sequence” refers to the sequence used as a basis for a sequence comparison; a reference sequence may be in another embodiment, a subset of a larger sequence, for example, as a segment of a full-length cDNA or gene sequence given in a sequence listing or may comprise a complete cDNA or gene sequence.

Therefore in this aspect of the invention and in one embodiment, the invention provide an isolated Inf-20 nucleic acid, encoding an apoptosis inhibition protein, induced by TNF-α or FADD, wherein said nucleic acid has a nucleic acid sequence having at least 67% similarity, or in another embodiment at least 85%, or in another embodiment, at least 95% or in another embodiment 99%, with the nucleic acid coding sequence of SEQ ID NO. 1.

In one embodiment, the invention provide an isolated Inf-20 nucleic acid, encoding an apoptosis regulation protein, induced by TNF-α or FADD, wherein said nucleic acid has a nucleic acid sequence having between about 67 to about 75% similarity, or in another embodiment, between about 75 to about 80% similarity, or in another embodiment, between about 80 to about 85% similarity, or in another embodiment, between about 85 to about 90% similarity, or in another embodiment, between about 90 to about 95% similarity, or in another embodiment, between about 95 to 100% similarity.

| hinf-20 Sequence (SEQ ID NO. 1) | |
|---|---|
| atggaatctt tttccagcaa atccctagcg ctccaagccg | 60 |
| aaaagaaact cttaagtaaa | |
| atggctggga gatcggtagc tcacctattc attgatgaaa | 120 |
| ccagctcaga ggtcctcgat | |
| gagctctatc gcgtttcgaa agagtacacg catagtagac | 180 |
| cacaagcaca acgggtgatc | |
| aaagacctga taaaggtagc agtcaaggtt gcagtacttc | 240 |
| accggaatgg atcttttgga | |
| ccgagcgaat tagcactggc gactcgtttt cgtcagaagt | 300 |
| tacgacaagg tgcgatgacg | |
| gctttgagtt tcggggaagt tgactttact ttcgaagctg | 360 |
| ccgtgcttgc gggtttgctt | |
| actgaatgcc gagacatact acttgagtta gtcgagcatc | 420 |
| acttaacacc caagtcacat | |
| ggtcgcatca ggcatgtgtt tgatcattat agtgatccag | 480 |
| gcctgttgac agccctttac | |
| ggacctgact tcacacagca cctagggaag atttgtgatg | 540 |
| gcctaaggaa attgctggac | |
| gagggaaaat tg | 552 |

It is to be understood by those skilled in the art and it is intended here, that when reference is made in one embodiment to a particular sequence listings, such reference includes sequences which substantially correspond to its complementary sequence and those described including allowances for minor sequencing errors, single base changes, deletions, substitutions and the like, such that any such sequence variation corresponds to the nucleic acid sequence of the pathogenic organism or disease marker to which the relevant sequence listing relates.

Optimal alignment of sequences for aligning a comparison window may be conducted by the local homology algorithm of Smith and Waterman (1981) Adv. Appl. Math. 2:482, by the homology alignment algorithm of Needleman and Wunsch (1970) J Mol. Biol. 48:443, by the search for similarity method of Pearson and Lipman (1988) Proc. Natl. Acad Sci. (USA) 85:2444, or by computerized implementations of these algorithms (GAP, BESTFIT, FASTA, and TFASTA in the Wisconsin Genetics Software Package Release 7.0, Genetics Computer Group, 575 Science Dr., Madison, Wisc.).

“Substantial identity” or “substantial sequence identity” mean in one embodiment that two peptide sequences, when optimally aligned, such as by the programs GAP or BESTFIT using default gap which share at least 90 percent sequence identity, preferably at least 95 percent sequence identity, more preferably at least 99 percent sequence identity or more. “Percentage amino acid identity” or “percentage amino acid sequence identity” refers to a comparison of the amino acids of two polypeptides which, when optimally aligned, have approximately the designated percentage of the same amino acids. For example, “95% amino acid identity” refers to a comparison of the amino acids of two polypeptides which when optimally aligned have 95% amino acid identity. Preferably, residue positions which are not identical differ by conservative amino acid substitutions. For example, the substitution of amino acids having similar chemical properties such as charge or polarity are not likely to effect the properties of a protein. Examples include glutamine for asparagine or glutamic acid for aspartic acid.

The percent identity between the two sequences is a function of the number of identical positions shared by the sequences (i.e., % identity of identical positions/total # of positions (e.g., overlapping) ×100). Preferably, the two sequences are the same length. The determination of percent homology between two sequences can be accomplished using a mathematical algorithm. A preferred, non-limiting example of a mathematical algorithm utilized for the comparison of two sequences is the algorithm of Karlin and Altschul (1990) Proc. Natl. Acad. Sci. USA 87:2264-2268, modified as in Karlin and Altschul (1993) Proc. Natl. Acad, Sci. USA 90:5873-5877. Such an algorithm is incorporated into the NBLAST and XBLAST programs of Altschul, et al. (1990) J. Mol. Biol. 215:403-410. BLAST nucleotide searches can be performed with the NBLAST program, score=100, wordlength=12 to obtain nucleotide sequences homologous to Inf-20 nucleic acid molecules of the invention. BLAST protein searches can be performed with the X13LAST program, score=50, wordlength=3 to obtain amino acid sequences homologous to Inf-20 protein molecules of the invention. To obtain gapped alignments for comparison purposes, Gapped BLAST can be utilized as described in Altschul et al., (1997) Nucleic Acids Res. :3389-3402. When utilizing BLAST and Gapped BLAST programs, the default parameters of the respective programs (e.g., X13LAST and NBLAST) can be used. See http://www.ncbi.nlm.nih.gov. Another preferred, non-limiting example of a mathematical algorithm utilized for the comparison of sequences is the algorithm of Myers and Miller, CABIOS 4:11-17 (1988). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used. The percent identity between two sequences can be determined using techniques similar to those described above, with or without allowing gaps. In calculating percent identity, only exact matches are counted.

In one embodiment, this invention provides a nucleic acid having a sequence complementary to the sequence of the isolated nucleic acid of the inf-20 gene. Specifically, this invention provides an oligonucleotide of at least 15 nucleotides capable of specifically hybridizing with a sequence of nucleotides present within a nucleic acid which encodes the inf-20. In one embodiment the nucleic acid is DNA or RNA. In another embodiment the oligonucleotide is labeled with a detectable marker. In another embodiment the oligonucleotide is a radioactive isotope, a fluorophor or an enzyme.

Oligonucleotides which are complementary may be obtained as follows: The polymerase chain reaction is then carried out using the two primers. Following PCR amplification, the PCR-amplified regions of a viral DNA can be tested for their ability to hybridize to the three specific nucleic acid probes listed above. Alternatively, hybridization of a viral DNA to the above nucleic acid probes can be performed by a Southern blot procedure without viral DNA amplification and under stringent hybridization conditions.

High stringent hybridization conditions are selected at about 5° C. lower than the thermal melting point (Tm) for the specific sequence at a defined ionic strength and pH. The Tm is the temperature (under defined ionic strength and pH) at which 50% of the target sequence hybridizes to a perfectly matched probe. Typically, stringent conditions will be those in which the salt concentration is at least about 0.02 molar at pH 7 and the temperature is at least about 60° C. As other factors may significantly affect the stringency of hybridization, including, among others, base composition and size of the complementary strands, the presence of organic solvents, ie. salt or formamide concentration, and the extent of base mismatching, the combination of parameters is more important than the absolute measure of any one.

Hybridization with moderate stringency may be attained for example by: 1) filter pre-hybridizing and hybridizing with a solution of 3× sodium chloride, sodium citrate (SSC), 50% formnamide, 0.1M Tris buffer at Ph 7.5, 5× Denhardt's solution; 2.) pre-hybridization at 37° C. for 4 hours; 3) hybridization at 37° C. with amount of labelled probe equal to 3,000,000 cpm total for 16 hours; 4) wash in 2×SSC and 0.1% SDS solution; 5) wash 4× for 1 minute each at room temperature at 4× at 60° C. for 30 minutes each; and 6) dry and expose to film.

The phrase “specifically hybridizing to” refers to a nucleic acid probe that hybridizes, 30 duplexes or binds only to a particular target DNA or RNA sequence when the target sequences are present in a preparation of total cellular DNA or RNA. By selectively hybridizing it is meant that a probe binds-to a given target in a manner that is detectable in a different manner from non-target sequence under high stringency conditions of hybridization. in a different “Complementary” or “target” nucleic acid sequences refer to those nucleic acid sequences which selectively hybridize to a nucleic acid probe. Proper annealing conditions depend, for example, upon a probe's length, base composition, and the number of mismatches and their position on the probe, and must often be determined empirically.

In one embodiment, a host cell is a fertilized oocyte or in another embodiment, an embryonic stem cell into which Inf-20-coding sequences have been introduced. Such host cells can then he used to create non-human transgenic animals in which exogenous Inf-20 sequences have been introduced into their genome or homologous recombinant animals in which endogenous Inf-20 sequences have been altered. Such animals are useful for studying the function and/or activity of Inf-20 and for identifying and/or evaluating modulators of Inf-20 activity.

Therefore, in this aspect of the invention and in one embodiment, the invention provide a transgenic, nonhuman mammal comprising the Inf-20 isolated nucleic acid

As used herein, a “transgenic animal’, is a non-human animal, which is in one embodiment, a mammal, or in another embodiment a rodent such as a rat or mouse, in which one or more of the cells of the animal includes a transgene. In another embodiment, transgenic animals include non-human primates, sheep, dogs, cows, goats, chickens, amphibians, etc.

Mutations can be made in a nucleic acid encoding Inf-20 such that a particular codon is changed to a codon which codes for a different amino acid but the TNF-α or FADD induced apoptosis inhibition and interaction with caspase-8 activity is maintained. Such a mutation is generally made by making the fewest nucleotide changes possible. A substitution mutation of this sort can be made to change an amino acid in the resulting protein in a non-conservative manner (i.e., by changing the codon from an amino acid belonging to a grouping of amino acids having a particular size or characteristic to an amino acid belonging to another grouping) or in a conservative manner (i.e., by changing the codon from an amino acid belonging to a grouping of amino acids having a particular size or characteristic to an amino acid belonging to the same grouping). Such a conservative change generally leads to less change in the structure and function of the resulting protein. A non-conservative change is more likely to alter the structure, activity or function of the resulting protein. The present invention should be considered to include sequences containing conservative changes which do not significantly alter the activity or binding characteristics of the resulting protein. Substitutes for an amino acid within the sequence may be selected from other members of the class to which the amino acid belongs. For example, the nonpolar (hydrophobic) amino acids include alanine, leucine, isoleucine, valine, proline, phenylalanine, tryptophan and methionine. Amino acids containing aromatic ring structures are phenylalanine, tryptophan, and tyrosine. The polar neutral amino acids include glycine, serine, threonine, cysteine, tyrosine, asparagine, and glutamine. The positively charged (basic) amino acids include arginine, lysine and histidine. The negatively charged (acidic) amino acids include aspartic acid and glutamic acid. Such alterations will not be expected to affect apparent molecular weight as determined by polyacrylamide gel electrophoresis, or isoelectric point. This isolated nucleic acid also encodes mutant Inf-20 or the wildtype protein.

The invention further provides a recombinant expression vector comprising a DNA is molecule of the invention cloned into the expression vector in an antisense orientation. That is, the DNA molecule is operably linked to a regulatory sequence in a manner which allows for expression (by transcription of the DNA molecule) of an RNA molecule which is antisense to inf-20 mRNA. Regulatory sequences operably linked to a nucleic acid cloned in the antisense orientation can be chosen which direct the continuous expression of the antisense RNA molecule in a variety of cell types, for instance viral promoters and/or enhancers, or regulatory sequences can be chosen which direct constitutive, tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see Weintraub et al. (Reviews—Trends in Genetics, Vol. 1(1) 1986).

This invention provides in one embodiment a replicable vector comprising the isolated nucleic acid molecule of the DNA virus. The vector includes, but is not limited to: a plasmid, cosmid, phage or yeast artificial chromosome (YAC) which contains at least a portion of the isolated nucleic acid molecule. As an example to obtain these vectors, insert and vector DNA can both be exposed to a restriction enzyme to create complementary ends on both molecules which base pair with each other and are then ligated together with DNA ligase. Alternatively, linkers can be ligated to the insert DNA which correspond to a restriction site in the vector DNA, which is then digested with the restriction enzyme which cuts at that site. Other means are also available and known to an ordinary skilled practitioner.

In this aspect of the invention and in one embodiment, the invention provides an isolated inf-20 polypeptide selected from a group of polypeptide, which are in one embodiment a polypeptide comprising the amino acid sequence as set forth in SEQ ID NO:2, or in another embodiment, a polypeptide comprising a naturally occurring amino acid sequence, which is at least 72% identical to the amino acid sequence as set forth in SEQ ID NO:2, wherein said polypeptide has TNF-α or FADD induced apoptosis inhibition activity and is able to interact with caspase-8, or in another embodiment, a biologically active fragment of a polypeptide having an amino acid sequence of SEQ ID NO:2, wherein said fragment has TNF-α or FADD induced apoptosis inhibition activity, or in another embodiment, a polypeptide encoded by the nucleic acid sequence as set forth in SEQ ID NO. 1.

In one embodiment, the invention provides a polypeptide comprising a naturally occurring amino acid sequence, which is between about 72 and 75% identical to the amino acid sequence as set forth in SEQ ID NO:2, or in another embodiment between about 75 and 80% identical to the amino acid sequence as set forth in SEQ ID NO:2, or in another embodiment between about 80 and 85% identical to the amino acid sequence as set forth in SEQ ID NO:2, or in another embodiment between about 85 and 90% identical to the amino acid sequence as set forth in SEQ ID NO:2, or in another embodiment between about 90 and 95% identical to the amino acid sequence as set forth in SEQ ID NO:2, or in another embodiment between about 95 and 100% identical to the amino acid sequence as set forth in SEQ ID NO:2.

hInf-20 Polypeptide Sequence (SEQ ID NO. 2):
Met-Glu-Ser-Phe-Ser-Ser-Lys-Ser-Leu-Ala-Leu-Gln-
Ala-Glu-Lys-Lys-Leu-Leu-Ser-Lys-Met-Ala-Gly-Arg-
Ser-Val-Ala-His-Leu-Phe-Ile-Asp-Glu-Thr-Ser-Ser-
Glu-Val-Leu-Asp-Glu-Leu-Tyr-Arg-Val-Ser-Lys-Glu-
Tyr-Thr-His-Ser-Arg-Pro-Gln-Ala-Gln-Arg-Val-Ile-
Lys-Asp-Leu-Ile-Lys-Val-Ala-Val-Lys-Val-Ala-Val-
Leu-His-Arg-Asn-Gly-Ser-Phe-Gly-Pro-Ser-Glu-Leu-
Ala-Leu-Ala-Thr-Arg-Phe-Arg-Gln-Lys-Leu-Arg-Gln-
Gly-Ala-Met-Thr-Ala-Leu-Ser-Phe-Gly-Glu-Val-Asp-
Phe-Thr-Phe-Glu-Ala-Ala-Val-Leu-Ala-Gly-Leu-Leu-
Thr-Glu-Cys-Arg-Asp-Ile-Leu-Leu-Glu-Leu-Val-Glu-
His-His-Leu-Thr-Pro-Lys-Ser-His-Gly-Arg-Ile-Arg-
His-Val-Phe-Asp-His-Tyr-Ser-Asp-Pro-Gly-Leu-Leu-
Thr-Ala-Leu-Tyr-Gly-Pro-Asp-Phe-Thr-Gln-His-Leu-
Gly-Lys-Ile-Cys-Asp-Gly-Leu-Arg-Lys-Leu-Leu-Asp-
Glu-Gly-Lys-Leu.

In another embodiment, this invention provides an antibody which specifically binds to; in one embodiment a polypeptide comprising the amino acid sequence as set forth in SEQ ID NO:2, or in another embodiment, a polypeptide comprising a naturally occurring amino acid sequence at least 72% identical to the amino acid sequence as set forth in SEQ ID NO:2, wherein said polypeptide has TNF-α or FADD induced apoptosis inhibition activity and is able to interact with caspase-8, or in another embodiment, a biologically active fragment of a polypeptide having an amino acid sequence of SEQ ID NO:2, wherein said fragment has TNF-α or FADD induced apoptosis inhibition activity, or in another embodiment, a polypeptide encoded by the nucleic acid sequence as set forth in SEQ ID NO. 1.

In one embodiment, this invention provides an antibody which specifically binds to a polypeptide comprising a naturally occurring amino acid sequence, which is between about 72 and 75% identical to the amino acid sequence as set forth in SEQ ID NO:2, or in another embodiment between about 75 and 80% identical to the amino acid sequence as set forth in SEQ ID NO:2, or in another embodiment between about 80 and 85% identical to the amino acid sequence as set forth in SEQ ID NO:2, or in another embodiment between about 85 and 90% identical to the amino acid sequence as set forth in SEQ ID NO:2, or in another embodiment between about 90 and 95% identical to the amino acid sequence as set forth in SEQ ID NO:2, or in another embodiment between about 95 and 100% identical to the amino acid sequence as set forth in SEQ ID NO:2.

In one embodiment the antibody is a monoclonal antibody. In another embodiment the antibody is a polyclonal antibody. The antibody or DNA molecule may be labelled with a detectable marker including, but not limited to: a radioactive label, or a colorimetric, a luminescent, or a fluorescent marker, or gold. Radioactive labels include, but are not limited to: ³H, ¹⁴C, ³²P, ³³P; ³⁵S, ³⁶Cl, ⁵¹Cr, ⁵⁷Co, ⁵⁹Co, ⁵⁹Fe, ⁹⁰Y, ¹²⁵I, ¹³¹I, and ¹⁸⁶Re. Fluorescent markers include but are not limited to: fluorescein, rhodamine and auramine. Colorimetric markers include, but are not limited to: biotin, and digoxigenin. Methods of producing the polyclonal or monoclonal antibody are known to those of ordinary skill in the art.

In another embodiment, the term “antibody” refers to intact antibodies and binding fragments thereof In one embodiment, fragments compete with the intact antibody from which they were derived for specific binding to an antigen. In another embodiment antibodies or binding fragments thereof, can be chemically conjugated to, or expressed as, fusion proteins with other proteins.

Further, the antibody or nucleic acid molecule complex may be detected by a second antibody which may be linked to an enzyme, such as alkaline phosphatase or horseradish peroxidase. Other enzymes which may be employed are well known to one of ordinary skill in the art.

In one embodiment, the term “Specifically binds to an antibody” or in another embodiment “specifically immunoreactive with”, when referring to a protein or peptide, refers to a binding reaction which is determinative of the presence of the Inf-20 of the invention in the presence of a heterogeneous population of proteins and other biologics including viruses other than the Inf-20. Thus, under designated immunoassay conditions, the specified antibodies bind to the Inf-20 antigens and do not bind in a significant amount to other antigens present in the sample. Specific binding to an antibody under such conditions may require an antibody that is selected for its specificity for a particular protein. For example, antibodies raised to the human Inf-20 immunogen described herein can be selected to obtain antibodies specifically immunoreactive with the Inf-20 proteins and not with other proteins. These antibodies recognize proteins homologous to the human Inf-20 protein. A variety of immunoassay formats may be used to select antibodies specifically immunoreactive with a particular protein. For example, solid-phase ELISA immunoassays are routinely used to select monoclonal antibodies specifically immunoreactive with a protein.

In one embodiment, the terms “amino acid” or “amino acid sequence,” refer to an oligopeptide, peptide, polypeptide, or protein sequence, or a fragment of any of these, and to naturally occurring or synthetic molecules. In another embodiment, the term “fragments,” “immunogenic fragments,” or “antigenic fragments” refer to fragments of inf-20 protein, which are about 5 to about 15 amino acids in length in one embodiment, and which retain some biological activity or immunological activity of inf-20. In one embodiment, “amino acid sequence” is recited herein to refer to an amino acid sequence of a naturally occurring protein molecule, or in another embodiment “amino acid sequence” and like terns are not meant to limit the amino acid sequence to the complete native amino acid sequence associated with the recited protein molecule.

This invention provides a method to select specific regions on the polypeptide encoded by the isolated DNA molecule of the DNA virus to generate antibodies. The protein sequence may be determined from the cDNA sequence. Amino acid sequences may be analyzed by methods well known to those skilled in the art to determine whether they produce hydrophobic or hydrophilic regions in the proteins which they build. In the case of cell membrane proteins, hydrophobic regions are well known to form the part of the protein that is inserted into the lipid bilayer of the cell membrane, while hydrophilic regions are located on the cell surface, in an aqueous environment. Usually, the hydrophilic regions will be more immunogenic than the hydrophobic regions. Therefore the hydrophilic amino acid sequences may be selected and used to generate antibodies specific to polypeptide encoded by the isolated nucleic acid molecule encoding the DNA virus. The selected peptides may be prepared using commercially available machines. As an alternative, DNA, such as a cDNA or a fragment thereof, may be cloned and expressed and the resulting polypeptide recovered and used as an immunogen.

Polyclonal antibodies against these peptides may be produced by immunizing animals using the selected peptides. Monoclonal antibodies are prepared using hybridoma technology by fusing antibody producing B cells from immunized animals with myeloma cells and selecting the resulting hybridoma cell line producing the desired antibody. Alternatively, monoclonal antibodies may be produced by in vitro techniques known to a person of ordinary skill in the art. These antibodies are useful to detect the expression of polypeptide encoded by the isolated DNA molecule of the DNA virus in living animals, in humans, or in biological tissues or fluids isolated from animals or humans.

The antibodies may be detectably labelled, utilizing conventional labelling techniques well-known to the art. Thus, the antibodies may be radiolabelled using, for example, radioactive isotopes such as ³H, ¹²⁵I, ¹³¹I, and ³⁵S. The antibodies may also be labelled using fluorescent labels, enzyme labels, free radical labels, or bacteriophage labels, using techniques known in the art. Typical fluorescent labels include fluorescein isothiocyanate, rhodamine, phycoerythrin, phycocyanin, alophycocyanin, and Texas Red.

Since specific enzymes may be coupled to other molecules by covalent links, the possibility also exists that they might be used as labels for the production of tracer materials. Suitable enzymes include alkaline phosphatase, beta-galactosidase, glucose-6-phosphate dehydrogenase, maleate dehydrogenase, and peroxidase. Two principal types of enzyme immunoassay are the enzyme-linked immunosorbent assay (ELISA), and the homogeneous enzyme immunoassay, also known as enzyme-multiplied immunoassay (EMIT, Syva Corporation, Palo Alto, Calif.). In the ELISA system, separation may be achieved, for example, by the use of antibodies coupled to a solid phase. The EMIT system depends on deactivation of the enzyme in the tracer-antibody complex; the activity can thus be measured without the need for a separation step.

Additionally, chemiluminescent compounds may be used as labels. Typical chemiluminescent compounds include luminol, isoluminol, aromatic acridinium esters, imidazoles, acridinium salts, and oxalate esters. Similarly, bioluminescent compounds may be utilized for labelling, the bioluminescent compounds including luciferin, luciferase, and aequorin. Once labeled, the antibody may be employed to identify and quantify immunologic counterparts (antibody or antigenic polypeptide) utilizing techniques well-known to the art.

In one embodiment, antibodies to the Inf-20 can be used to detect the agent in the sample. In brief, to produce antibodies to the agent or peptides, the sequence being targeted is expressed in transfected cells, preferably bacterial cells, and purified. The product is injected into a mammal capable of producing antibodies. Either monoclonal or polyclonal antibodies (as well as any recombinant antibodies) specific for the gene product can be used in various immunoassays. Such assays include competitive immunoassays, radioimmunoassays, Western blots, ELISA, indirect immunofluorescent assays and the like.

Monoclonal antibodies or recombinant antibodies may be obtained by various techniques familiar to those skilled in the art. Briefly, spleen cells or other lymphocytes from an animal immunized with a desired antigen are immortalized, commonly by fusion with a myeloma cell. Alternative methods of immortalization include transformation with Epstein Barr Virus, oncogenes, or retroviruses, or other methods well known in the art. Colonies arising from single immortalized cells are screened for production of antibodies of the desired specificity and affinity for the antigen, and yield of the monoclonal antibodies produced by such cells may be enhanced by various techniques, including injection into the peritoneal cavity of a vertebrate host. New techniques using recombinant phage antibody expression systems can also be used to generate monoclonal antibodies.

Such peptides may be produced by expressing the specific sequence in a recombinantly engineered cell such as bacteria, yeast, filamentous fungal, insect (especially employing baculoviral vectors), and mammalian cells. Those of skill in the art are knowledgeable in the numerous expression systems available for expression of herpes virus protein.

Briefly, the expression of natural or synthetic nucleic acids encoding viral protein will typically be achieved by operably linking the desired sequence or portion thereof to a promoter (which is either constitutive or inducible), and incorporated into an expression vector. The vectors are suitable for replication or integration in either prokaryotes or eukaryotes. Typical cloning vectors contain antibiotic resistance markers, genes for selection of transformants, inducible or regulatable promoter regions, and translation terminators that are useful for the expression of viral genes.

Methods for the expression of cloned genes in bacteria are also well known. In general, to obtain high level expression of a cloned gene in a prokaryotic system, it is advisable to construct expression vectors containing a strong promoter to direct mRNA transcription. The inclusion of selection markers in DNA vectors transformed in E. coli is also useful. Examples of such markers include genes specifying resistance to antibiotics.

The peptides derived form the nucleic acids; peptide fragments are produced by recombinant technology may be purified by standard techniques well known to those of skill in the art. Recombinantly produced sequences can be directly expressed or expressed as a fusion protein. The protein is then purified by a combination of cell lysis (e.g., sonication) and affinity chromatography. For fusion products, subsequent digestion of the fusion protein with an appropriate proteolytic enzyme releases the desired peptide.

In another embodiment, this invention encompasses peptide fragments of the polypeptide which result from proteolytic digestion products of the polypeptide. In another embodiment, the derivative of the polypeptide has one or more chemical moieties attached thereto. In another embodiment the chemical moiety is a water soluble polymer. In another embodiment the chemical moiety is polyethylene glycol. In another embodiment the chemical moiety is mon-, di-, tri- or tetrapegylated. In another embodiment the chemical moiety is N-terminal monopegylated.

A polypeptide “fragment,” “portion” or “segment” is a stretch of amino acid residues of at least about five to seven contiguous amino acids, or in another embodiment at least about seven to nine contiguous amino acids, or in another embodiment at least about nine to 13 contiguous amino acids and, or in another embodiment, at least about 20 to 30 or more contiguous amino acids.

According to this aspect of the invention and in one embodiment, the following fragments are considered part of the present invention.

Murine Inf-20 (SEQ ID NO.3)

| | |
|---|---|
| M E S E S S K S L A L Q A E K K L L | (SEQ ID NO. 3) |
| S K M A G R S V A H L F I D E T S S | |
| E V L D E L Y R V S K E Y T H S R P | |
| K A Q R V I K D L I K V A V K V A V | |
| L H R S G C F G P G E L A L A T R F | |
| R Q K L R Q G A M T A L S F G E V D | |
| F T F E A A V L A G L L V E C R D I | |
| L L E L V E H H L T P K S H D R I R | |
| H V F D H Y S D P D L L A A L Y G P | |
| D F T Q H L G K I C D G L R K L L D | |
| E G K L. | |

100% Identity Fragment mInf-20-hInf-20

| | |
|---|---|
| MESFSSKSLALQAEKKLLSKMAGRSVAHLFIDETSSEVLDELYRVSKEYTHSRP | (SEQ ID NO. 4) |

100% Identity Fragment mInf-20-hInf-20

(SEQ ID NO. 5)

ELALATRFRQKIRQGAMTALSFGEVDFTFEAAVLAGLL

100% Identity Fragment mInf-20-hInf-20

| | |
|---|---|
| AQRVIKDLIKVAVKVAVLHR | (SEQ ID NO. 6) |

100% Identity Fragment mInf-20-hInf-20

| | |
|---|---|
| ECRDILLELVEHHLTPKSH | (SEQ ID NO. 7) |

100% Identity Fragment mInf-20-hInf-20

| | |
|---|---|
| ALYGPDFTQHLGKICDGLRKLLD | (SEQ ID NO. 8) |

100% Identity Fragment mInf-20-hInf-20

| | |
|---|---|
| RIRHVFDHYSDP | (SEQ ID NO. 9) |

100% Identity fragment hInf-20-ssc-c2DED

| | |
|---|---|
| TSSEVLDELYRV | (SEQ ID NO. 10) |

It should be noted that all amino-acid residue sequences are represented herein by formulae whose left and right orientation is in the conventional direction of amino-terminus to carboxy-terminus. Furthermore, it should be noted that a dash at the beginning or end of an amino acid residue sequence indicates a peptide bond to a further sequence of one or more amino-acid residues.

Synthetic polypeptide, prepared using the well known techniques of solid phase, liquid phase, or peptide condensation techniques, or any combination thereof, can include natural and unnatural amino acids. Amino acids used for peptide synthesis may be standard Boc (N-amino protected N-t-butyloxycarbonyl) amino acid resin with the standard deprotecting, neutralization, coupling and wash protocols of the original solid phase procedure of Merrifield (1963, J. Am. Chem. Soc. 85:2149-2154), or the base-labile N-amino protected 9-fluorenylmethoxycarbonyl (Fmoc) amino acids first described by Carpino and Han (1972, J. Org. Chem. 37:3403-3409). Thus, polypeptide of the invention may comprise D-amino acids, a combination of D- and L-amino acids, and various “designer” amino acids (e.g., methyl amino acids, C-methyl amino acids, and N-methyl amino acids, etc.) to convey special properties. Synthetic amino acids include ornithine for lysine, fluorophenylalanine for phenylalanine, and norleucine for leucine or isoleucine. Additionally, by assigning specific amino acids at specific coupling steps, alpha-helices, alpha turns, beta sheets, beta-turns, and cyclic peptides can be generated.

In one aspect of the invention, the peptides may comprise a special amino acid at the C-terminus which incorporates either a CO₂H or CONH₂ side chain to simulate a free glycine or a glycine-amide group. Another way to consider this special residue would be as a D or L amino acid analog with a side chain consisting of the linker or bond to the bead. In one embodiment, the pseudo-free C-terminal residue may be of the D or the L optical configuration; in another embodiment, a racemic mixture of D and L-isomers may be used.

In an additional embodiment, pyroglutamate may be included as the N-terminal residue of the peptide. Although pyroglutamate is not amenable to sequence by Edman degradation, by limiting substitution to only 50% of the peptides on a given bead with N-terminal pyroglutamate, there will remain enough non-pyroglutamate peptide on the bead for sequencing. One of ordinary skill would readily recognize that this technique could be used for sequencing of any peptide that incorporates a residue resistant to Edmnan degradation at the N-terminus. Other methods to characterize individual peptides that demonstrate desired activity are described in detail infra. Specific activity of a peptide that comprises a blocked N-terminal group, e.g., pyroglutamate, when the particular N-terminal group is present in 50% of the peptides, would readily be demonstrated by comparing activity of a completely is (100%) blocked peptide with a non-blocked (0%) peptide.

In addition, the present invention envisions preparing peptides that have more well defined structural properties, and the use of peptidomimetics, and peptidomimetic bonds, such as ester bonds, to prepare peptides with novel properties. In another embodiment, a peptide may be generated that incorporates a reduced peptide bond, i.e., R₁—CH₂—NH—R₂, where R₁ and R₂ are amino acid residues or sequences. A reduced peptide bond may be introduced as a dipeptide subunit. Such a molecule would be resistant to peptide bond hydrolysis, e.g., protease activity. Such peptides would provide ligands with unique function and activity, such as extended half-lives in vivo due to resistance to metabolic breakdown, or protease activity. Furthermore, it is well known that in certain systems constrained peptides show enhanced functional activity (Hruby, 1982, Life Sciences 31:189-199; Hruby et al., 1990, Biochem J. 268:249-262); the present invention provides a method to produce a constrained peptide that incorporates random sequences at all other positions.

A constrained, cyclic or rigidized peptide may be prepared synthetically, provided that in at least two positions in the sequence of the peptide an amino acid or amino acid analog is inserted that provides a chemical functional group capable of cross-linking to constrain, cyclise or rigidize the peptide after treatment to form the cross-link. Cyclization will be favored when a turn-inducing amino acid is incorporated. Examples of amino acids capable of cross-linking a peptide are cysteine to form disulfide, aspartic acid to form a lactone or a lactase, and a chelator such as carboxyl-glutamic acid (Gla) (Bachem) to chelate a transition metal and form a cross-link. Protected carboxyl glutamic acid may be prepared by modifying the synthesis described by Zee-Cheng and Olson (1980, Biophys. Biochem. Res. Commun. 94:1128-1132). A peptide in which the peptide sequence comprises at least two amino acids capable of cross-linking may be treated, e.g., by oxidation of cysteine residues to form a disulfide or addition of a metal ion to form a chelate, so as to cross-link the peptide and form a constrained, cyclic or rigidized peptide.

The present invention provides strategies to systematically prepare cross-links. For example, if four cysteine residues are incorporated in the peptide sequence, different protecting groups may be used (Hiskey, 1981, in The Peptides: Analysis, Synthesis, Biology, Vol. 3, Gross and Meienhofer, eds., Academic Press: New York, pp. 137-167; Ponsanti et al., 1990, Tetrahedron 46:8255-8266). The first pair of cysteine may be deprotected and oxidized, then the second set may be deprotected and oxidized. In this way a defined set of disulfide cross-links may be formed. Alternatively, a pair of cysteine and a pair of collating amino acid analogs may be incorporated so that the cross-links are of a different chemical nature.

The following non-classical amino acids may be incorporated in the peptide in order to introduce particular conformational motifs: 1,2,3,4-tetrahydroisoquinoline-3-carboxylate (Kazmierski et al., 1991, J. Am. Chem. Soc. 113:2275-2283); (2S,3S)-methyl-phenylalanine, (2S,3R)-methyl-phenylalanine, (2R,3S)-methyl-phenylalanine and (2R,3R)-methyl-phenylalanine (Kazmierski and Hruby, 1991, Tetrahedron Lett.); 2-aminotetrahydronaphthalene-2-carboxylic acid (Landis, 1989, Ph.D. Thesis, University of Arizona); hydroxy-1,2,3,4-tetrahydroisoquinoline-3-carboxylate (Miyake et al., 1989, J. Takeda Res. Labs. 43:53-76);-carboline (D and L) (Kazmierski, 1988, Ph.D. Thesis, University of Arizona); HIC (histidine isoquinoline carboxylic acid) (Zechel et al., 1991, Int. J. Pep. Protein Res. 43); and HIC (histidine cyclic urea) (Dharanipragada).

The following amino acid analogs and peptidomimetics may be incorporated into a peptide to induce or favor specific secondary structures: LL-Acp (LL-3-amino-2-propenidone-6-carboxylic acid), a dipeptide analog (Kemp et al., 1985, J. Org. Chem. 50:5834-5838); and analogs provided by the following references: Nagai and Sato, 1985, Tetrahedron Lett. 26:647-650; DiMaio et al., 1989, J. Chem. Soc. Perkin Trans. p. 1687; also a Gly-Ala turn analog (Kahn et al., 1989, Tetrahedron Lett. 30:2317); amide bond isostere (Jones et al., 1988, Tetrahedron Lett. 29:3853-3856); tretrazol (Zabrocki et al., 1988, J. Am. Chem. Soc. 110:5875-5880); DTC (Samanen et al., 1990, Int. J. Protein Pep. Res. 35:501:509); and analogs taught in Olson et al., 1990, J. Am. Chem. Sci. 112:323-333 and Garvey et al., 1990, J. Org. Chem. 56:436. Conformationally restricted mimetics of beta turns and beta bulges, and peptides containing them, are described in U.S. Pat. No. 5,440,013, issued Aug. 8, 1995 to Kahn.

The present invention further provides for modification or derivatization of the polypeptide or peptide of the invention. Modifications of peptides are well known to one of ordinary skill, and include phosphorylation, carboxymethylation, and acylation. Modifications may be effected by chemical or enzymatic means. In another aspect, glycosylated or fatty acylated peptide derivatives may be prepared. Preparation of glycosylated or fatty acylated peptides is well known in the art. Fatty acyl peptide derivatives may also be prepared. For example, and not by way of limitation, a free amino group (N-terminal or lysyl) may be acylated, e.g., myristoylated. In another embodiment an amino acid comprising an aliphatic side chain of the structure —(CH₂)ₙCH₃ may be incorporated in the peptide. This and other peptide-fatty acid conjugates suitable for use in the present invention are disclosed in U.K. Patent GB-8809162.4, International Patent Application PCT/AU89/00166.

In accordance with the present invention there may be employed conventional molecular biology, microbiology, and recombinant DNA techniques within the skill of the art. Such techniques are explained fully in the literature. See, e.g., Sambrook et al, “Molecular Cloning: A Laboratory Manual” (1989); “Current Protocols in Molecular Biology” Volumes I-III [Ausubel, R. M., ed. (1994)]; “Cell Biology: A Laboratory Handbook” Volumes I-III [J. E. Celis, ed. (1994))]; “Current Protocols in Immunology” Volumes I-III [Coligan, J. E., ed. (1994)]; “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

Chemical Moieties For Derivatization Chemical moieties suitable for derivatization may be selected from among water soluble polymers. The polymer selected should be water soluble so that the component to which it is attached does not precipitate in an aqueous environment, such as a physiological environment. Preferably, for therapeutic use of the end-product preparation, the polymer will be pharmaceutically acceptable. One skilled in the art will be able to select the desired polymer based on such considerations as whether the polymer/component conjugate will be used therapeutically, and if so, the desired dosage, circulation time, resistance to proteolysis, and other considerations. For the present component or components, these may be ascertained using the assays provided herein.

The water soluble polymer may be selected from the group consisting of, for example, polyethylene glycol, copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols and polyvinyl alcohol. Polyethylene glycol propionaldenhyde may have advantages in manufacturing due to its stability in water.

The number of polymer molecules so attached may vary, and one skilled in the art will be able to ascertain the effect on function. One may mono-derivatize, or may provide for a di-, tri-, tetra- or some combination of derivatization, with the same or different chemical moieties (e.g., polymers, such as different weights of polyethylene glycols). The proportion of polymer molecules to component or components molecules will vary, as will their concentrations in the reaction mixture. In general, the optimum ratio (in terms of efficiency of reaction in that there is no excess unreacted component or components and polymer) will be determined by factors such as the desired degree of derivatization (e.g., mono, di-, tri-, etc.), the molecular weight of the polymer selected, whether the polymer is branched or unbranched, and the reaction conditions.

In one embodiment, the compositions of the invention described hereinbelow are any of the polypeptides, or in another embodiment peptides or other similarly appropriate molecules described hereinabove.

As discussed above this invention provides in one embodiment, novel compounds that have biological properties useful for the treatment of disorders mediated by caspases, and in particular those mediated by apoptotic caspases and induced by TNF-α or FADD. In one embodiment, the compounds are useful for the treatment of disorders resulting from an overactive apoptotic response. In Another embodiment, the compounds of the invention are useful for the treatment of stroke, traumatic, brain injury, spinal cord injury, meningitis, Alzheimer's disease, Parkinson's disease, Huntington's disease, Kennedy's disease, prion disease, multiple sclerosis, spinal muscular atrophy, myocardial infarction, congestive heart failure and various other forms of acute and chronic heart disease, atherosclerosis, aging, burns, organ transplant rejection, graft versus host disease, hepatitis-B, -C, -G, various forms of liver disease including acute alcoholic hepatitis, yellow fever, dengue fever, Japanese encephalitis, glomerulonephritis, renal disease, H. pylori-associated gastric and duodenal ulcer disease, HIV infection, tuberculosis, alopecia, diabetes, sepsis, Shigellosis, uveitis, inflammatory peritonitis, pancreatitis, erythematosus, scleroderma, chronic thyroiditis, Graves' disease, autoimmune gastritis, autoimmune hemolytic anemia, autoimmune neutropenia, thrombocytopenia, HIV-related encephalitis, myasthenia gravis, small bowel ischemia in disease or post-surgery, psoriasis, atopic dermatitis, myelodysplatic syndrome, acute and chronic myelogenous leukemia, metastatic melanoma, Kaposi's sarcoma, and Wiscott-Aldrich syndrome. Accordingly, in another aspect of the present invention, compositions are provided, which comprise any one of the compounds described herein (or a prodrug, pharmaceutically acceptable salt or other pharmaceutically acceptable derivative thereof), and optionally comprise a pharmaceutically acceptable carrier. In certain embodiments, these compositions optionally further comprise one or more additional therapeutic agents. Alternatively, a compound of this invention may be administered to a patient in need thereof in combination with the administration of one or more other therapeutic agents. For example, in one embodiment, additional therapeutic agents for conjoint administration or inclusion in a pharmaceutical composition with a compound of this invention may be an approved β-INF agent, or agents undergoing approval in the Food and Drug Administration, or other similarly situated authority worldwide that ultimately obtain approval for the treatment of any disorder resulting from a TNF-α or FADD induced, or caspase-mediated disorder or, in other embodiments, from an inappropriate apoptotic response. It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or a pro-drug or other adduct or derivative of a compound of this invention which upon administration to a patient in need is capable of providing, directly or indirectly, a compound as otherwise described herein, or a metabolite or residue thereof.

Therefore, in this aspect of the invention and in one embodiment, the invention provides a composition, comprising a inf-20 encoded polypeptide, represented by SEQ ID NO. 1 and a pharmaceutically acceptable carrier, excipient, flow agent, processing aid, diluent or a combination thereof.

In one embodiment, the composition further comprises a carrier, excipient, lubricant, flow aid, processing aid or diluent, wherein said carrier, excipient, lubricant, flow aid, processing aid or diluent is a gum, starch, a sugar, a cellulosic material, an acrylate, calcium carbonate, magnesium oxide, talc, lactose monohydrate, magnesium stearate, colloidal silicone dioxide or mixtures thereof.

In one embodiment, the composition further comprises a binder, a disintegrant, a buffer, a protease regulator, a surfactant, a solubilizing agent, a plasticizer, an emulsifier, a stabilizing agent, a viscosity increasing agent, a sweetner, a film forming agent, or any combination thereof.

In one embodiment, the composition is a particulate composition coated with a polymer (e.g., poloxamers or poloxamines). Other embodiments of the compositions of the invention incorporate particulate forms protective coatings, protease regulators or permeation enhancers for various routes of administration, including parenteral, pulmonary, nasal and oral. In one embodiment the pharmaceutical composition is administered parenterally, paracancerally, transmucosally, transdermally, intramuscularly, intravenously, intradermally, subcutaneously, intravaginally, intraperitonealy, intraventricularly, intracranially or intratumorally.

In one embodiment, the compositions of this invention may be in the form of a pellet, a tablet, a capsule, a solution, a suspension, a dispersion, an emulsion, an elixir, a gel, an ointment, a cream, or a suppository.

In another embodiment, the composition is in a form suitable for oral, intravenous, intraaorterial, intramuscular, subcutaneous, parenteral, transmucosal, transdermal, or topical administration. In one embodiment the composition is a controlled release composition. In another embodiment, the composition is an immediate release composition. In one embodiment, the composition is a liquid dosage form. In another embodiment, the composition is a solid dosage form.

In one embodiment, the term “pharmaceutically acceptable carriers” includes, but is not limited to, may refer to 0.01-0.1M and preferably 0.05M phosphate buffer, or in another embodiment 0.8% saline. Additionally, such pharmaceutically acceptable carriers may be in another embodiment aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media.

In one embodiment, the compounds of this invention may include compounds modified by the covalent attachment of water-soluble polymers such as polyethylene glycol, copolymers of polyethylene glycol and polypropylene glycol, carboxymethyl cellulose, dextran, polyvinyl alcohol, polyvinylpyrrolidone or polyproline are known to exhibit substantially longer half-lives in blood following intravenous injection than do the corresponding unmodified compounds (Abuchowski et al., 1981; Newmark et al., 1982; and Katre et al., 1987). Such modifications may also increase the compound's solubility in aqueous solution, eliminate aggregation, enhance the physical and chemical stability of the compound, and greatly reduce the immunogenicity and reactivity of the compound. As a result, the desired in vivo biological activity may be achieved by the administration of such polymer-compound abducts less frequently or in lower doses than with the unmodified compound.

The pharmaceutical preparations of the invention can be prepared by known dissolving, mixing, granulating, or tablet-forming processes. For oral administration, the active ingredients, or their physiologically tolerated derivatives in another embodiment, such as salts, esters, N-oxides, and the like are mixed with additives customary for this purpose, such as vehicles, stabilizers, or inert diluents, and converted by customary methods into suitable forms for administration, such as tablets, coated tablets, hard or soft gelatin capsules, aqueous, alcoholic or oily solutions. Examples of suitable inert vehicles are conventional tablet bases such as lactose, sucrose, or cornstarch in combination with binders such as acacia, cornstarch, gelatin, with disintegrating agents such as cornstarch, potato starch, alginic acid, or with a lubricant such as stearic acid or magnesium stearate.

Examples of suitable oily vehicles or solvents are vegetable or animal oils such as sunflower oil or fish-liver oil. Preparations can be effected both as dry and as wet granules. For parenteral administration (subcutaneous, intravenous, intraarterial, or intramuscular injection), the active ingredients or their physiologically tolerated derivatives such as salts, esters, N-oxides, and the like are converted into a solution, suspension, or emulsion, if desired with the substances customary and suitable for this purpose, for example, solubilizers or other auxiliaries. Examples are sterile liquids such as water and oils, with or without the addition of a surfactant and other pharmaceutically acceptable adjuvants. Illustrative oils are those of petroleum, animal, vegetable, or synthetic origin, for example, peanut oil, soybean oil, or mineral oil. In general, water, saline, aqueous dextrose and related sugar solutions, and glycols such as propylene glycols or polyethylene glycol are preferred liquid carriers, particularly for injectable solutions.

In addition, the composition can contain minor amounts of auxiliary substances such as wetting or emulsifying agents, pH buffering agents which enhance the effectiveness of the active ingredient.

An active component can be formulated into the composition as neutralized pharmaceutically acceptable salt forms. Pharmaceutically acceptable salts include the acid addition salts (formed with the free amino groups of the polypeptide or antibody molecule), which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The compositions of the present invention are formulated in one embodiment for oral delivery, wherein the active compounds may be incorporated with excipients and used in the form of ingestible tablets, buccal tables, troches, capsules, elixirs, suspensions, syrups, wafers, and the like. The tablets, troches, pills, capsules and the like may also contain the following: a binder, as gum tragacanth, acacia, cornstarch, or gelatin; excipients, such as dicalcium phosphate; a disintegrating agent, such as corn starch, potato starch, alginic acid and the like; a lubricant, such as magnesium stearate; and a sweetening agent, such as sucrose, lactose or saccharin may be added or a flavoring agent, such as peppermint, oil of wintergreen, or cherry flavoring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. Syrup of elixir may contain the active compound sucrose as a sweetening agent methyl and propylparabens as preservatives, a dye and flavoring, such as cherry or orange flavor. In addition, the active compounds may be incorporated into sustained-release, pulsed release, controlled release or postponed release preparations and formulations.

Controlled or sustained release compositions include formulation in lipophilic depots (e.g. fatty acids, waxes, oils). Also comprehended by the invention are particulate compositions coated with polymers (e.g. poloxamers or poloxamines) and the compound coupled to antibodies directed against tissue-specific receptors, ligands or antigens or coupled to ligands of tissue-specific receptors.

In one embodiment, the composition can be delivered in a controlled release system. For example, the agent may be administered using intravenous infusion, an implantable osmotic pump, a transdermal patch, liposomes, or other modes of administration. In one embodiment, a pump may be used (see Langer, supra; Sefton, CRC Crit. Ref. Biomed. Eng. 14:201 (1987); Buchwald et al., Surgery 88:507 (1980); Saudek et al., N. Engl. J. Med. 321:574 (1989). In another embodiment, polymeric materials can be used. In another embodiment, a controlled release system can be placed in proximity to the therapeutic target, i.e., the brain, thus requiring only a fraction of the systemic dose (see, e.g., Goodson, in Medical Applications of Controlled Release, supra, vol. 2, pp. 115-138 (1984). Other controlled release systems are discussed in the review by Langer (Science 249:1527-1533 (1990).

Such compositions are in one embodiment liquids or lyophilized or otherwise dried formulations and include diluents of various buffer content (e.g., Tris-HCl., acetate, phosphate), pH and ionic strength, additives such as albumin or gelatin to prevent absorption to surfaces, detergents (e.g., Tween 20, Tween 80, Pluronic F68, bile acid salts), solubilizing agents (e.g., glycerol, polyethylene glycerol), anti-oxidants (e.g., ascorbic acid, sodium metabisulfite), preservatives (e.g., Thimerosal, benzyl alcohol, parabens), bulking substances or tonicity modifiers (e.g., lactose, mannitol), covalent attachment of polymers such as polyethylene glycol to the protein, complexation with metal ions, or incorporation of the material into or onto particulate preparations of polymeric compounds such as polylactic acid, polglycolic acid, hydrogels, etc., or onto liposomes, microemulsions, micelles, unilamellar or multilamellar vesicles, erythrocyte ghosts, or spheroplasts. Such compositions will influence the physical state, solubility, stability, rate of in vivo release, and rate of in vivo clearance. Controlled or sustained release compositions include formulation in lipophilic depots (e.g., fatty acids, waxes, oils). Also comprehended by the invention are particulate compositions coated with polymers (e.g., poloxamers or poloxamines). Other embodiments of the compositions of the invention incorporate particulate forms, protective coatings, protease regulators, or permeation enhancers for various routes of administration, including parenteral, pulmonary, nasal, and oral. In one embodiment, the pharmaceutical composition is administered parenterally, transmucosally, transdermally, intramuscularly, intravenously, intradermally, subcutaneously, intraperitonealy, intraventricularly, or intracranially.

In another embodiment, the compositions of this invention comprise one or more, pharmaceutically acceptable carrier materials.

In one embodiment, the carriers for use within such compositions are biocompatible, and in another embodiment, biodegradable. In other embodiments, the formulation may provide a relatively constant level of release of one active component. In other embodiments, however, a more rapid rate of release immediately upon administration may be desired. In other embodiments, release of active compounds may be event-triggered. The events triggering the release of the active compounds may be the same in one embodiment, or different in another embodiment. Events triggering the release of the active components may be exposure to moisture in one embodiment, lower pH in another embodiment, or temperature threshold in another embodiment. The formulation of such compositions is well within the level of ordinary skill in the art using known techniques. Illustrative carriers useful in this regard include microparticles of poly(lactide-co-glycolide), polyacrylate, latex, starch, cellulose, dextran and the like. Other illustrative postponed-release carriers include supramolecular biovectors, which comprise a non-liquid hydrophilic core (e.g., a cross-linked polysaccharide or oligosaccharide) and, optionally, an external layer comprising an amphiphilic compound, such as phospholipids. The amount of active compound contained in one embodiment, within a sustained release formulation depends upon the site of administration, the rate and expected duration of release and the nature of the condition to be treated suppressed or inhibited.

In one embodiment, the compositions of the invention are used for treatment, or in another embodiment, prevention, or in another embodiment, inhibition or in another embodiment, alleviating symptoms associated with a neurodegenerative disease, wherein, in another embodiment, the neurodegenerative disease is Multiple Sclerosis (MS).

This invention also provides in one embodiment, methods of use of the compositions mentioned hereinabove.

In one embodiment, the invention provides a method for identifying, diagnosing or predicting a candidate for developing a neurodegenerative disease comprising determining whether inf-20 is overexpressed as compared with a standard. In another embodiment Inf-20 is overexpressed by at least 25%, as compared with a standard.

In another embodiment, the term “expression” refers to the transcription and stable accumulation of sense (mRNA) or antisense RNA derived from the nucleic acid fragment or fragments of the invention. Expression may refer in one embodiment, to translation of mRNA into a polypeptide.

In one embodiment, the term “overexpressed” refers to the results obtained upon differentially determining expression Inf-20 or its products, when compared to a standard. The profile is assigned to a given subject, which reflects comparative results between his or her expression as compared to a standard. In one embodiment, the expression profile further comprises a determination of relative expression of nucleic acids, which do not code for a functional protein, as compared to the standard.

The term “differentially expressed” refers to a relative abundance or absence of expression in a subject as compared to a standard. Differential expression refers to changed expression, either higher or lower, in the subject, as compared to the standard.

Differential gene expression may include in one embodiment, a comparison of expression between two or more genes, or in another embodiment, a comparison of the ratios of the expression between two or more genes, or in another embodiment, a comparison of two differently processed products of the same gene, which differ between control subjects and subjects in which transplant was rejected, or in another embodiment, in the same subject pre- and post transplantation. Differential expression refers in one embodiment to quantitative, as well as in another embodiment, qualitative, differences in the temporal or cellular expression pattern in a gene or its expression products as described herein.

In one embodiment, a gene expression is analyzed using a sample of peripheral blood of a subject being evaluated. In another embodiment, a tissue biopsy serves as the source for evaluation, or in another embodiment, the diseased tissue, whose treatment is desired is used as the source for gene expression profile.

The gene expression analyzed in the methods of this invention will comprise a gene differentially expressed in a healthy subject, as compared to those diagnosed as prone to contract a neurodegenerative disease. The pattern of the differentially expressed gene will comprise increased expression of Inf-20 gene, in subjects more likely to develop a neurodegenerative disease, whereas the reverse profile is more predictive of a subject not likely to develop such a disease.

In one embodiment, determining the gene expression refers to methods to assess mRNA abundance, or in another embodiment, gene product abundance. According to this aspect of the invention, and in one embodiment, gene product refers to the translated protein. In one embodiment, protein abundance reflects gene expression profiles, which may be determined, in other embodiments, by any methods known in the art, such as, but not limited to Western blot analysis, RIA, ELISA, HPLC, functional assays, such as enzymatic assays, as applicable, and others. In one embodiment, expression profile is determined by a change in mRNA levels, or in another embodiment in surface expression, or in another embodiment in secretion or in another embodiment other partitioning of a polypeptide.

In another embodiment, the expression is a relative value as compared to a standard. In one embodiment the term “standard” may refer to a pooled sample of subject exhibiting complete remission for the same neurodegenerative disease. In another embodiment, standard may be ethnically- or gender- or age-matched recipients. It is to be understood that the standard may be derived from any subject, or pool of subjects, whose gene expression profile or profiles, once generated, is sufficient to detect even minute relative differences in expression, when compared to a potential candidate of developing a neurodegenerative disease.

In one embodiment, “increased expression” refers to an increase in the level or in another embodiment, activity of target gene product relative to the level or activity of target gene product in a standard. In another embodiment, increased expression refers to between a 10 to about a 250% increase in mRNA levels, or in another embodiment, in protein levels. In another embodiment, increased expression refers to changes in gene expression at the mRNA or protein level, in terms of its pattern of expression in particular examples, such as, for example, and in one embodiment, increased expression in tissue, but not in the blood, for example, in damaged tissue for which the transplant is required. In one embodiment, increased expression is synonymous with overexpression, or stimulated expression. In another embodiment, increased expression is a relative determination, wherein expression is greater than the standard, or in cases where expression is absent in the standard, this despite expression being barely detectable in the subject. It is to be understood that any such circumstance described hereinabove, represents increased expression for the methods of this invention.

In one embodiment, “compared to a standard”, refers to relative changes in expression where the standard is derived from a single individual, or is derived from pooled subjects who have successfully undergone treatment for a neurodegenerative disease. In another embodiment, a standard can be derived from a single subject following about 1 to about 5 years of having undergone successful treatment of the neurodegenerative disease, which in another embodiment is multiple sclerosis (MS).

In one embodiment, increases or decreases in gene expression are tissue specific, and encompass, in other embodiments, post-transcriptional and/or post-translational modifications of the gene products, which result in differences in non-modified gene expression.

In one embodiment, the results obtained are compared to a standard, which, in another embodiment, may comprise a series of standards, which, in another embodiment is used in the methods of the invention for quantification of differential expression of Inf-20. In one embodiment, the standard may comprise any embodiment listed herein, and in another embodiment, will be suitable for a particular application of the methods described herein. In one embodiment, the standard comprises nucleic acids when the method is used for the determination of nucleic acid profile, or in another embodiment the standard is a protein when the method is used for the determination of expressed protein profile.

In one embodiment, detecting differential expression of Inf-20, is done via the methods of the invention and is accomplished using establsihed PCR, ELISA, RIA, and other similarly recognized methods, and the reagents comprise those appropriate for the particular assay for detection.

In one embodiment, the invention provides a method reducing symptoms of a neurodegenerative disease in a subject, comprising administering to said subject the compositions of the invention as described herein, in an amount effective to reduce symptoms of said neurodegenerative disease. In another embodiment, the neurodegenerative disease is multiple sclerosis (MS).

The clinical course of MS is highly variable and unpredictable, with many patients experiencing acute episodes of exacerbations, followed by periods of remission. The disease progresses at various paces to a chronic, degenerative condition. Frequently, a diagnosis of MS may not be made for many years after the onset of symptoms because the symptoms can be variable, sporadic, and similar to those associated with other disorders. As the disease progresses, patients are frequently unable to remain fully ambulatory, and their functional systems steadily decline. The most severe cases of MS are characterized by paralysis or even death. MS may occur in several forms classified as primary progressive, relapsing-remitting, and secondary progressive, depending on the pathophysiology, progression and severity of the symptoms. In one embodiment, the claimed methods can be used in reducing the symptoms, or in another embodiment, in treating or in another embodiment in preventing, or in another embodiment in inhibiting the neurodegenerative disease of patients suffering from MS at any time in the progression of the disease, and may be used for patients suffering from primary progressive MS in one embodiment, or secondary progressive MS in another embodiment, or relapsing remitting MS in another embodiment.

In another embodiment, the term “preventing” refers to decreasing the probability that an organism contracts or develops an abnormal condition, including any disease or disorder described herein.

Parkinson's disease (PD) is a common disabling disease of old age affecting about one percent of the population over the age of 60 in the United States. The disease is pathologically associated with degeneration within the nuclear masses of the extrapyramidal system and the characteristic apoptosis of melanin-containing cells in the substantia nigra and a corresponding reduction in dopamine release from the corpus striatum, leading to severe imbalance of dopamine/acetylcholine in the brain. Dopamine acts in the brain as a neurotransmitter in the synaptic cleft of the neurons promoting signal transmission from one cell to another. PD is a progressivly degenerative desease characterized among other symptoms, by muscle rigidity, coarse tremors (especially of the hands) and postural deformity. In in one embodiment, symptoms at a severity of the United Parkinson's disease Rating Scale (UPDRS) is used to gauge efficiency according to the methods of this invention.

In one embodiment, administration in cycles refers to the steps of providing compositions of this invention for a specified period of time, ceasing the administration, and readministering the compositions of this invention, for a second period of time. In one embodiment, the steps are repeated and are dependent upon the severity of symptoms.

In another embodiment, the methods in this invention are for diseases, which are acute. In one embodiment, a single administration of the compositions of the invention is administered, or in another embodiment, the administration is for the duration of the acute phase of the disease. In another embodiment, the administration is for the duration of the disease, and a prescribed period following the disease, whether the disease is acute or chronic.

In one embodiment, the administration of the compositions of the invention, serves to prevent or treat relapse of a neurodegenerative disease. In another embodiment, the administration of the compositions of the invention, serves to delay the onset of the neurodegenerative disease, or in another embodiment, reduce its severity.

In one embodiment, a neurological disease is the neurodegenerative disease. In one embodiment, the neurodegenerative disease is Parkinson's disease (PD), Alzheimer's Disease (AD), Dementia with Lewy Bodies (DLB) or multi-infract Dementia, inappropriate sleepiness in narcolepsy.

In one embodiment, the term “effective amount” refers to an amelioration of symptoms, abrogation of symptoms, halting of disease pathogenesis, altering of disease time course, shortening of symptomatic stages, prevention of relapse, lengthening of time between relapsing and remitting stages of the disease, decreasing relapse frequency, enhancing mental function/acuity, concentration, cognitive abilities, eliminating side-effects or a combination thereof.

In one embodiment, the term “treating” includes preventative treatment, or in another embodiment disorder-remitative treatment, or in another embodiment palliative treatment.

In one embodiment, the term “inhibiting” refers to the lessening, or in another embodiment decreasing disease, or in another embodiment increasing the latency period between progression of disease stages as described herein, or in another embodiment affecting partial remission, or in another embodiment, complete remission, or in another embodiment stopping disease progression.

In one embodiment, the term “progression” refers to increasing in scope, or in another embodiment severity, or in another embodiment advancing, or in another embodiment growing or or in another embodiment becoming worse. In one embodiment. The term “recurrence” refers in one embodiment to the return of a disease after a remission.

In one embodiment, the term “administering” refers to bringing a subject in contact with a compound or composition of the present invention. Administration can be accomplished, in one embodiment in vitro, i.e. in a test tube, or in another embodiment in vivo, i.e. in cells or tissues.

In one embodiment, the beneficial effect of the methods and compositions of the invention is pharmacokinetic or pharmacodynamic co-action resulting from the combination of therapeutic agents. Wherein simultaneous may refer to administration of base within a composition, administration of 2 or more compositions via the same route of administration, or in another embodiment, via different route. In another embodiment, administration is with a single capsule having a fixed ratio of each therapeutic agent or in another embodiment multiple, or in another embodiment single capsules for each of the therapeutic agents.

In one embodiment sequential or in another embodiment substantially simultaneous administration of each therapeutic agent can be effected in one embodiment, by any appropriate route including, but not limited to, oral routes, intravenous routes, intramuscular routes, and direct absorption through mucous membrane, cutaneous or subcutaneous tissues. In another embodiment, the therapeutic agents according to the invention can be administered by the same route or by different routes. In one embodiment, a first therapeutic agent of the combination selected may be administered by intravenous injection while the other therapeutic agents of the combination may be administered orally. Alternatively, in another embodiment, all therapeutic agents may be administered orally or all therapeutic agents may be administered by intravenous injection in another embodiment.

In one embodiment the invention provides a medium having disposed thereon an oligonucleotide-hybridized cRNA of inf-20, which oligonucleotide may be detectibly labeled, where, in another embodiment, the medium is machine-readable, or in another embodiment, the machine readable form is a microarray chip.

In one embodiment the term “microarray” refers to a spatially defined pattern of oligonucleotide probes on a solid support. “solid support” refers in one embodiment to a fixed organizational support matrix, such as silica, polymeric materials, or glass. In another embodiment, at least one surface of the substrate is partially planar. In one embodiment, it is desirable to physically separate regions of the substrate to delineate synthetic regions, such as, in one embodiment, with trenches, grooves, wells or the like. In another embodiment, solid substrates may refer to slides, beads and chips.

In one embodiment, the medium further comprises cRNA of the sequence as set forth in SEQ ID. No. 1. As would be apreciated by one skilled in the art, cRNA of the sequence as set forth in SEQ ID. No. 1, could be generated by numerous known methods.

In one embodimnent, cRNA refers to complementary ribonucleic acid or substantially complementary ribonucleic acid. In another embodiment, cRNA refers to the hybridization or base pairing between nucleotides or nucleic acids, such as, for instance, between the two strands RNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair in one embodiment, with at least about 70% of the nucleotides of the other strand, or in another embodiment with about 90% to 95%, and in another embodiment with about 98 to 100%.

In one embodiment, the invention provide a method of regulating death receptor induced apoptosis in a subject, comprising administering to said subject the compositions of the invention, in an amount effective to regulate death receptor induced apoptosis in said subject. In one embodiment, the apoptosis is induecd by TNF-α, or in another embodiment, by FADD.

In another embodiment, the term “death receptor” refers to a member of the TNFR superfamily, which transmit death-inducing signals via the so-called “death domain” (DD). After activation and recruitment of intracellular adapter proteins, they initiate a signaling cascade that activates caspases and finally executes cell death.

In one embodiment, the term “domain” refers to a structural part of a biomolecule that contributes to a known or suspected function of the biomolecule. Domains may be co-extensive with regions or portions thereof; domains may also incorporate a portion of a biomolecule that is distinct from a particular region, in addition to all or part of that region.

The TF-α/TNF-β receptor families (TNFR) consist of approximately 20 ligands and 30 receptors. These proteins play crucial roles in regulating lymphocyte activation and apoptosis, and therefore, are important for immunity and inflammation. Several TNF receptor family members (often called death receptors) possess intracellular death domains (DDs), allowing association with other DD-containing adaptor proteins such as FADD and TRADD. FADD contains both a DD and a death effector domain (DED). The DED domain is used to interact with other DED-containing proteins such as caspase-8, which is essential for initiating the caspase cascade. In addition to inducing apoptosis, most death receptors can also, paradoxically, promote cell survival. As mentioned herein, death adapter proteins such as FADD can interact with caspases through analogous DED-DED or caspase recruitment domain (CARD)-CARD association. To transmit apoptosis signals, the death effector domain (DED) of FADD recruits procaspase 8, which contains two DED and a caspase domain consisting of the large subunit (p20) and the small subunit (p10). Dimerization of procaspase 8 facilitates its self-processing and subsequent generation of active heterotetramers (p20/p10). The active caspase 8 initiates apoptosis by the cleavage of downstream substrates such as procaspase 3 in one embodiment.

In another embodiment Inf-20 binds directly to caspase-8, thereby blocking apoptosis by directly acting on caspases. As mentioned herein, death adapter proteins such as FADD can interact with caspases through analogous DED-DED or caspase recruitment domain (CARD)-CARD association. Since Inf-20 has a putative DED domain, Inf-20 evidently interacts with FADD in one embodiment, or caspase-8 in another embodiment, or c-FLIP_{L} in another embodiment, via DED domain, thereby regulating death receptor induced apoptosis.

It is to be understood by a person skilled in the art, that the methods of the invention are to be used similarly on a single cell in one embodiment, or on a group of cells. The methods of the invention are envisioned in another embodiment, to be carried out in-vitro, or in another embodiment in-situ, or in another embodiment, in-vivo.

Therefore, according to this aspect of the invention, and in one embodiment, the invention provides a method of regulating death receptor induced apoptosis in a cell, comprising contacting said cell with the compositions of the invention, in an amount effective to regulate death receptor induced apoptosis in the cell as described herein for a subject In one embodiment, the apoptosis is induecd by TNF-α or in another embodiment, by FADD.

In one embodiment, the term “contacting” refers to bringing together, either directly or indirectly, a compound into physical proximity to a polypeptide or polynucleotide of the invention. The polypeptide or polynucleotide can be in any number of buffers, salts, solutions etc. Contacting includes, for example, placing the compound into a beaker, microtiter plate, cell culture flask, or a microarray, such as a gene chip, or the like, which contains a nucleic acid molecule or polypeptide of the invention.

In one embodiment, the term “regulate” refers to a change in the activity of Inf-20-encoded protein. For example, regulation may cause an increase or a decrease in protein activity, binding characteristics, or any other biological, functional, or immunological properties of Inf-20.

In another embodiment, the term “activity” refers to a variety of measurable indicia suggesting or revealing binding, either direct or indirect; affecting a response, i.e. having a measurable affect in response to some exposure or stimulus, including, for example, the affinity of a compound for directly binding a polypeptide or polynucleotide of the invention, or, for example, measurement of amounts of upstream or downstream proteins or other similar functions after some stimulus or event, or enzymatic activity such as proteolysis of a substrate

In another embodiment, the invention provide a method of promoting cell survival, comprising increasing the expression of inf-20, wherein increasing said expression of said inf-20 is by contacting said cell with an inf-20 agonist.

In one embodiment, The nucleotide and amino acid sequence information provided by the present invention also makes it possible for the identification of binding compounds with which Inf-20 polypeptides or polynucleotides will interact. Methods to identify binding partner compounds include in another embodiment solution assays, or in another embodiment, in vitro assays wherein Inf-20 polypeptides are immobilized, and cell based assays in another embodiment. Identification of binding compounds of Inf-20 polypeptides provides candidates for therapeutic or prophylactic intervention in pathologies associated with normal or aberrant biological activity of Inf-20.

The invention includes several methods for identifying binding partners for Inf-20 polypeptides. In one embodiment, methods of the invention comprise the steps of (a) contacting a sample comprising the polypeptide of the invention with a compound, and (b) determining if TNF-α or FADD induced apoptosis inhibition activity in said sample is increased in comparison to a control sample lacking said compound. Determining if TNF-α or FADD induced apoptosis inhibition activity in said sample can be achieved by isolating the Inf-20 polypeptide/compound complex, and separating the Inf-20 polypeptide from the binding compound. An additional step of characterizing the physical, biological, and/or biochemical properties of the binding partner compound is also comprehended in another embodiment of the invention. In one embodiment, the Inf-20 polypeptide/partner complex is isolated using an antibody immunospecific for either the Inf-20 polypeptide or the candidate binding partner compound. In another embodiment, determining if TNF-α or FADD induced apoptosis inhibition activity in said sample can be carried out by using the methods described herein in Example 1.

According to this aspect of the invention and in one embodiment, the invention provide a method of screening a compound for effectiveness as an agonist of a polypeptide of the invention as described herein, the method comprising; contacting a sample comprising the polypeptide of the invention to a compound, and determining if TNF-α or FADD induced apoptosis inhibition activity in said sample is increased in comparison to a control sample lacking said compound. Similarly, and in another embodiment, the invention provide a method of screening a compound for its effectiveness as an antagonist of a polypeptide of the invention.

In one embodiment, the term “agonist” refers to a molecule which, when bound to Inf-20-encoded protein in one embodiment or a cell expressing Inf-20 in another embodiment, increases in one embodiment or prolongs in another embodiment, the duration of the effect of Inf-20 -encoded protein. Agonists may include in one embodiment proteins, nucleic acids, carbohydrates, or in another embodiment, molecules which bind to and modulate the effect of Inf-20.

In another embodiment, the term “antagonist,” refers to a molecule which, when bound to Inf-20, decreases the amount or the duration of the effect of the biological or immunological activity of Inf-20. Antagonists may include proteins, nucleic acids, carbohydrates, antibodies, or any other molecules which decrease the effect of Inf-20.

In another embodiment, either the Inf-20 polypeptide or the candidate binding compound comprises a label in one embodiment or tag in another embodiment, which facilitates its isolation, and methods of the invention to identify binding partner compounds include a step of isolating the Inf-20 polypeptide/compound complex through interaction with the label or tag. An exemplary tag is in one embodiment, a poly-histidine sequence, generally around six histidine residues, that permits isolation of a compound so labeled using nickel chelation. In another embodiment, other labels and tags, such as the FLAG.RTM. tag (Eastman Kodak, Rochester, N.Y.), well known and routinely used in the art, are within the scope of the invention.

In one embodiment, the invention also encompasses high throughput screening (HTS) assays to identify compounds that interact with or inhibit biological activity (i.e., inhibit enzymatic activity, binding activity, etc.) of a Inf-20 polypeptide. HTS assays permit screening of large numbers of compounds in an efficient manner. Cell-based HTS systems are contemplated to investigate the interaction between Inf-20 polypeptides and their binding partners. HTS assays are designed to identify “hits” or “lead compounds” having the desired property, from which modifications can be designed to improve the desired property. Chemical modification of the “hit” or “lead compound” is based in another embodiment on an identifiable structure/activity relationship between the “hit” and Inf-20 polypeptides.

Another aspect of the present invention is directed to methods of identifying compounds that bind to either a Inf-20 polypeptide or nucleic acid molecules encoding a Inf-20 polypeptide, comprising contacting a Inf-20 polypeptide, or a nucleic acid molecule encoding the same, with a compound, and determining whether the compound binds the Inf-20 polypeptide or a nucleic acid molecule encoding the same. Binding can be determined by binding assays which are well known to the skilled artisan, including, but not limited to, gel-shift assays, Western blots, radiolabeled competition assay, phage-based expression cloning, co-fractionation by chromatography, co-precipitation, cross linking, interaction trap/two-hybrid analysis, southwestern analysis, ELISA, and the like, which are described in, for example, Current Protocols in Molecular Biology (1999) John Wiley & Sons, NY, which is incorporated herein by reference in its entirety. The compounds to be screened (which may include compounds which are suspected to bind a Inf-20 polypeptide, or a nucleic acid molecule encoding the same) include, but are not limited to, extracellular, intracellular, biologic or chemical origin. The methods of the invention also embrace ligands including substrates, adaptor or receptor molecules that are attached to a label, such as a radiolabel (e.g., i¹²⁵, S³⁵, P³², P³³, H³), a fluorescence label, a chemiluminescent label, an enzymic label or an immunogenic label. Modulators falling within the scope of the invention include, but are not limited to, non-peptide molecules such as non-peptide mimetics, non-peptide allosteric effectors, and peptides. The Inf-20 polypeptide or polynucleotide employed in such a test may either be free in solution, attached to a solid support, borne on a cell surface or located intracellularly or associated with a portion of a cell. One skilled in the art can, for example, measure the formation of complexes between a Inf-20 polypeptide and the compound being tested. Alternatively, one skilled in the art can examine the diminution in complex formation between a Inf-20 polypeptide and its substrate caused by the compound being tested.

The present invention is particularly useful for screening compounds by using the Inf-20 polypeptides in any of a variety of drug screening techniques. The compounds to be screened (which may include compounds which are suspected to bind a Inf-20 polypeptide, or a nucleic acid molecule encoding the same) include, but are not limited to, extracellular, intracellular, biologic or chemical origin. The Inf-20 polypeptide or polynucleotide employed in such a test may either be free in solution, attached to a solid support, borne on a cell surface or located intracellularly or associated with a portion of a cell. One skilled in the art can, for example, measure the formation of complexes between a Inf-20 polypeptide and the compound being tested. Alternatively, one skilled in the art can examine the diminution in complex formation between a Inf-20 polypeptide and its substrate caused by the compound being tested.

The early biochemical events resulting in apoptosis induction by ligand-induced death receptor cross-linking are caused in one embodiment, by death-inducing signaling complex (DISC). Cross-linking of CD95 in one embodiment, or the two apoptosis-inducing TRAIL receptors in another embodiment, results in the recruitment of Fas-associated death domain (FADD) and caspase-8 to the DISC. In an embodiment of such interaction, the death domain of FADD binds to the death domain of CD95. The death effector domain (DED) of FADD in turn interacts with the death effector domain of procaspase 8 and thereby recruits this proenzyme to the DISC. Procaspase 8 is proteolytically cleaved and thereby activated at the DISC. Activated caspase-8 then initiates the apoptosis-executing caspase cascade. This cascade is further controlled by feedback processes between the intrinsic (mitochondrial) and extrinsic (death receptor) cell death pathways, thereby modulating the outcome of death receptor inducement.

Accordingly, in this aspect of the invention and in one embodiment, the invention provide a method of blocking apoptosis in a cell comprising contacting said cell with the polypeptide of the invention, wherein said polypeptide binds to caspase-8, therefore blocking apoptosis by directly acting on caspases.

In one embodiment, the invention provides a method of inhibiting apoptosis in a cell, the method comprising increasing the expression of Inf-20 in that cell. In one embodiment, the apoptosis is induced by TNF-α, or in another embodiment, apoptosis is induced by FADD. In one embodiment, the increase in expression of Inf-20, is done by using and agonist, which in another embodiment, was identified by the methods of the invention as described herein.

The following examples are presented in order to more fully illustrate the preferred embodiments of the invention. They should in no way be construed, however, as limiting the broad scope of the invention.

### EXAMPLES

### Example 1

### Identification of a Novel Death Effector Domain (DED)-Containing Protein that is Upregulated During Inflammation

### MATERIALS AND METHODS

Mice

C57BL/6 (B6) mice, 6-7 weeks of age, were purchased from Jackson Laboratory (Bar Harbor, Me.), and housed in the University of Pennsylvania Animal Care Facilities under pathogen-free conditions. All procedures used were pre-approved by the Institutional Animal Care and Use Committee.

Induction and Clinical Evaluation of EAE

For the induction of EAE, mice received 1) a subcutaneous injection on flanks of 300 μg myelin oligodendrocyte glycoprotein (MOG) peptide 38-50 in 0.1 ml PBS emulsified in an equal volume of complete Freund's adjuvant containing 5 mg/ml of mycobacterium tuberculosis H37RA (Difco, St. Louis, Mo.), and 2) an intravenous injection of 100 ng pertussis toxin in 0.1 ml PBS. A second injection of pertussis toxin (100 ng per mouse) was given 48 hours later. Mice were examined daily for signs of EAE and scored as follows: 0—no disease; 1—tail paralysis; 2—hind limb weakness; 3—hind limb paralysis; 4—hind limb plus forelimb paralysis; 5—moribund or dead.

Cell Lines, Expression Vector, and Reagents

Human embryonic kidney 293T cells, human cervical carcinoma HeLa cells, and murine fibroblast 3T3 cells were cultured in complete Dulbecco's modified Eagle's medium (DMEM). Constructs were generated using the pRK5 expression vector system. Anti-FLAG HRP antibody was purchased from Upstate Biotechnology (Lake placid, N.Y.) and anti-HA polyclonal antibody from Santa Cruz Biotech (Santa Cruz, Calif.). Protein-G-Agarose was purchased from Invitrogen (Carlsbad, Calif.).

Cloning of Inf-20

An expressed sequence tag (EST, AI847688) was obtained from the microarray analysis. A putative mouse full-length sequence was generated by aligning the EST sequence with the mouse EST database using the BLAST program. This gave rise to a novel gene of 555 bp (Gene bank accession number AF548004). Full length Inf-20 was then isolated from the spinal cord of the EAE mice by RT-PCR using Inf-20-specific primers. A human testis cDNA clone that bears a high homology with mouse Inf-20 was identified by BLAST search of the human EST database, and purchased directly from ATCC (gene bank accession number BG258888, clone number 4509736). The full-length Inf-20 was sequenced and cloned into the expression vector pRK5 with FLAG or HA tag at the C-terminus.

Northern Blotting Analysis

Total RNA from normal and EAE mouse tissues was prepared using the Trizol reagent (GIBCOL BRL, Carlsbad, Calif.), fractionated by electrophoresis, transferred to a nylon membrane and hybridized at 60° C. with ³²P-labeled DNA probes specific for Inf-20 or GAPDH.

Transient Transfection, Co-Immunoprecipitation, and Western Blotting Analysis

293T cells (1.5×10⁶) were seeded in an 100 mm plate and transfected with the expression vector pRK5 or recombinant vectors containing FLAG- or HA-tagged Inf-20 using the Fugene 6 reagent (Roche, Indianapolis, Ind.). Twenty to twenty-four hours after the transfection, cells were lysed with 1 ml lysis buffer in the present of protease regulator (50 mM HEPES, pH7.0, 150 mM NaCl, 1 mM EDTA, 0.1% NP-40, and 1 mM Na₃VO₄.) and immunoprecipitated with 5 μg anti-FLAG antibody for 3 hours and 25 μL protein-G-agarose beads for an additional 3 hours. The precipitates were resolved by SDS polyacrylamide gel electrophoresis (SDS-PAGE) and analyzed by immunoblotting with anti-FLAG or anti-HA antibodies.

To test TNF-induced apoptosis, HeLa cells were seeded in six-well plates (2×10⁵ cells/well) and transiently transfected with pRK5 vector or FLAG-tagged Inf-20 together with 20 ng of pEGFP-N3 plasmids as reporter plasmids. Twenty-four hours after transfection, cells were switched to DMEM containing 1% fetal bovine serum and cultured for 1 hour. TNF-α (50 ng/ml) and cycloheximide (10 μ/ml) were then added to the culture and cells were incubated for an additional 8-10 hours. All cells including those in suspension were collected, washed in staining buffer (0.5% BAS and 0.1% NaN₃ in PBS), and stained with propidium iodide (50 μg/ml) in the presence of RNase A (50 μg/ml). The percentage of apoptotic cells was determined by flow cytometry through gating the PI positive and EGFP positive cells. To test the role of Inf-20 in FADD-induced apoptosis, HeLa cells were co-transfected with 0.5 μg of Inf-20, 0.5 μg of FADD and 0.1 μg pCMV-LacZ expression plasmids. Twenty-four hours after transfection, cells were fixed with 0.5% glutaraldehyde and stained with the X-gal solution (35 mM K₃Fe(CN)₆, 35 mM K₄Fe(CN)₆-3H₂O, 1 mM MgCl₂, and 1 mg/ml X-gal in PBS). Apoptotic cells were identified based on their morphological characteristics. The percentage of apoptotic cells was calculated as follows: (number of apoptotic β-galactosidase positive cells/the total number of β-galactosidase positive cells)×100%.

Luciferase Reporter Assay

293T cells, 1×10⁶ cells/plate, were cultured at 37° C. for 24 hours. Different amounts of Inf-20 were used to co-transfect the cells with 20 ng p(κB)₃—INF—LUC and 25 ng pCMV-LacZ using the Fugene 6 reagent (Roche, Indianapolis, Ind.). Twenty-four hours after the transfection, cells were harvested and lysed in 300 μl of lysis buffer (Promega, Madison, Wisc.). Twenty microliters of the cell extract from each sample were tested for the luciferase activity using the Promega luciferase system according to the manufacturer's protocol (Promega, Madison, Wisc.). β-galactosidase activity was used as the internal control to monitor the efficiency of the transfection. For the β-galactosidase assay, 50 μl of the cell extract was incubated with 0.5 ml of Z buffer (60 mM Na₂HPO₄, 40 mM NaH₂PO₄, 10 mM KCl, 1 mM MgSO₄, 50 mM β-mecaptoethenol) and 0.1 ml of 4 mg/ml O-nitrophenyl-β-D-galactopyranoside at 30° C. for 30 min. The reaction was terminated by adding 0.25 ml of 1 M Na₂CO₃. The absorbance was measured at 420 nm on a Beckman Du 640 spectrophotometer.

RT-PCR

HeLa and NIH3T3 cells were seeded in 6-well plates and cultured in DMEM containing 10% FBS for 20-24 hours. Cells were then switched to serum-free medium and cultured for an additional 2 hours. Different amounts of TNF-α (Collaborative Biomedical products, Bedford, Mass.) were added to the cultures and cells were incubated for 4 hours. Total RNA was then isolated and purified using the RNeasy Mini-kit (Qiagen, Valencia, Calif.). RT-PCR was carried out using TNF specific primers.

### RESULTS

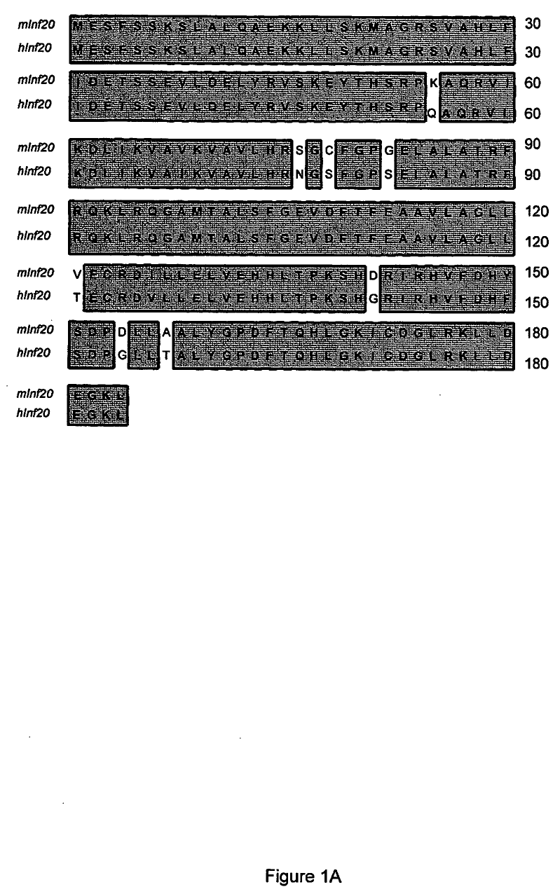

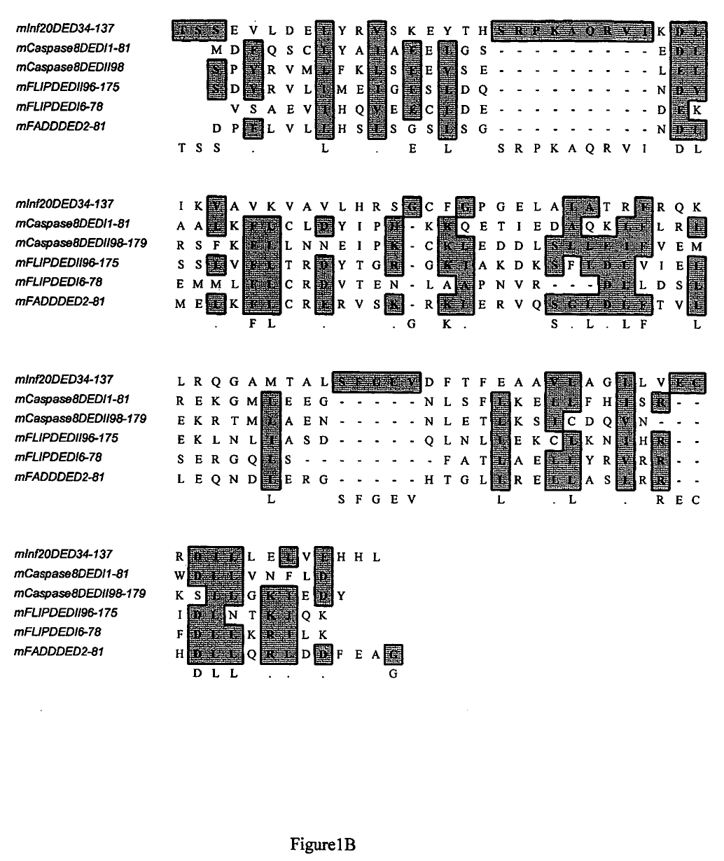

To determine the effect of inflammation on global gene expression in the CNS, C57BL/6 mice were first immunized with murine MOG peptide and sacrificed during the acute phase of EAE or during disease recovery. The acute phase of EAE is defined as the first EAE attack in mice leading to a clinical score of 3 or 4 (see Materials and Methods for disease score criteria, in the Induction and clinical evaluation of EAE section). Disease recovery is defined as a decrease of 2 graduations in clinical score for 5 or more consecutive days. Non-immunized naive mice were used as normal controls. Total RNA was isolated from the spinal cords and analyzed using the Affymetrix U74A and U74B chips as described in Materials and Methods. Two independent experiments were performed to ensure the reproducibility of the results. Genes whose expression levels were reproduced in both experiments (which accounted for >98% of the total genes tested) were subjected to a hierarchical clustering analysis to separate genes into clusters based on the similarity of expression across samples. This led to the identification of several clusters of genes whose expression was dramatically upregulated in the spinal cord of mice with EAE. One of these clusters contained several apoptosis-related genes including caspases 8, 11 and 12, and granzymes B and F as well as an expressed sequence tag (EST) that has a gene bank accession number of AI847688. By aligning this EST against the mouse EST database in the gene bank using the BLAST sequence homology search program, we identified a putative full-length cDNA, designated as Inf-20, with a predicted open reading frame (ORF) encoding 184 amino acids (FIG. 1A). By aligning this sequence against the human EST database in the gene bank using the same BLAST program, we identified a putative Inf-20 human homologue that shares 94% amino acid sequence identity with mouse Inf-20 (FIG. 1A). Human Inf-20 shares approximately 53% identity and 78% similarity with SSC-S2, a human TNF-inducible protein, that regulates apoptosis. Additionally, Inf-20 contains a putative DED domain that shows moderate identity/similarity to other known DED sequences (FIG. 1B/C). Specifically, the identity/similarity shared between DED of murine Inf-20 and those of the following proteins are as follows: SSC-S2, 50%/73%; murine cFLIP DED I, 19%/32%; murine cFLIP DED II, 13%/33%; murine caspase-8 DED I, 19%/40%; murine caspase-8 DED II, 20%/38%. The Inf-20 DED domain resides in its NH₂-terminal region (FIG. 1).

The chromosome location of Inf-20 was then determined by aligning the mouse and human Inf-20 sequences with mouse and human genome databases, respectively. A single locus was identified for mouse Inf-20 on chromosome III (3fl-3f3) and for human Inf-20 on chromosome I (1q21.2-1q21.3).

### Example 2

### Inf-20 is Expressed in Selected Tissues and Can be Induced by TNF

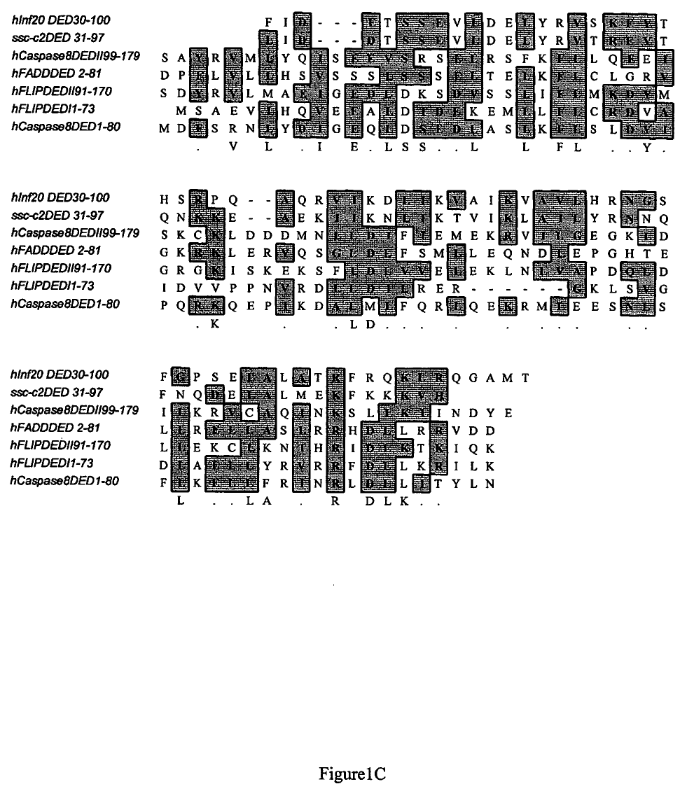

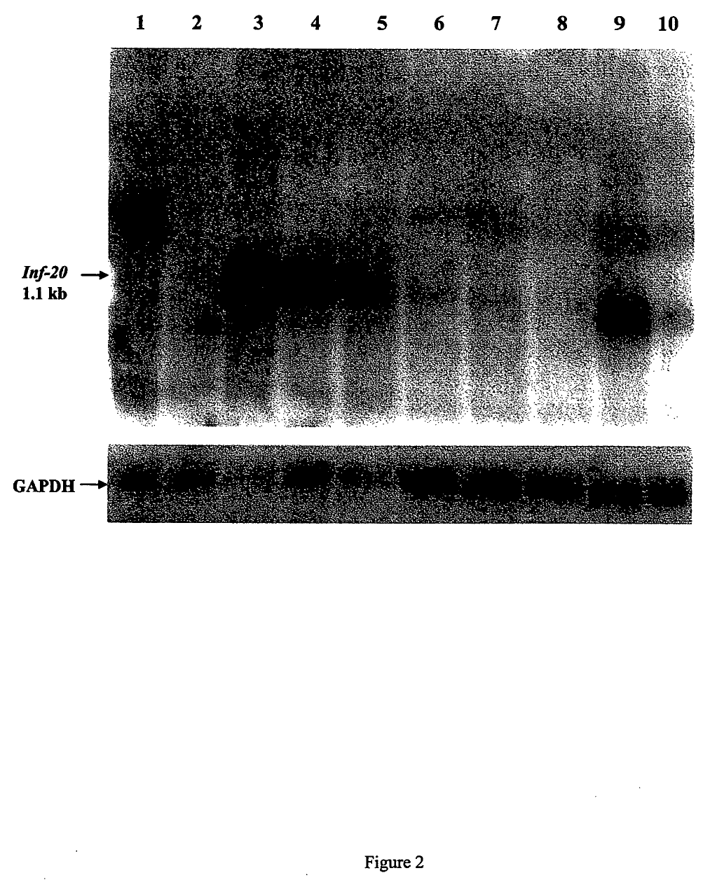

To determine the expression pattern of Inf-20, total RNA was extracted from different tissues of normal and EAE mice, fractionated by electrophoresis and transferred to Hybond-N nylon membrane. The RNA was then hybridized with the full-length Inf-20 is cDNA probe. A ˜1.1 kb transcript was detected in the thymus, spleen, lung, but not in the liver, heart, spinal cord or brain of normal mice. By contrast, high levels of Inf-20 mRNA were detected in the spinal cord and brain of EAE mice (FIG. 2). A higher molecular weight transcript was also detected in the kidney of normal mice and spinal cord of EAE mice (FIG. 2). Thus, Inf-20 is constitutively expressed in selected tissues and is upregulated during inflammation.

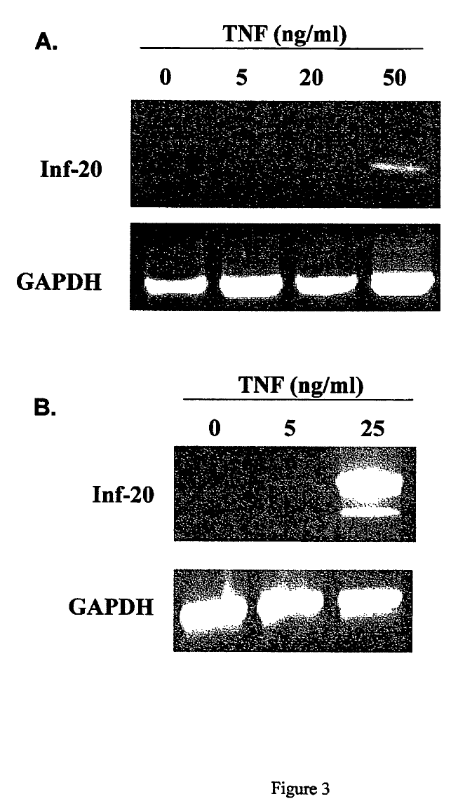

To determine whether Inf-20 can be activated by cytokines that mediate inflammation, human 293 cells and mouse NIH3T3 cells were treated with different amounts of TNF-α and examined Inf-20 gene expression by RT-PCR. Neither 293 cells nor NIH3T3 cells were found to constitutively expressed Inf-20. However, pre-incubation of these cells with TNF induced significant levels of Inf-20 gene expression (FIG. 3) demonstrating that Inf-20 is a TNF-α-inducible gene.

### Example 3

### Inf-20 Protein Specifically Interacts with Caspase-8

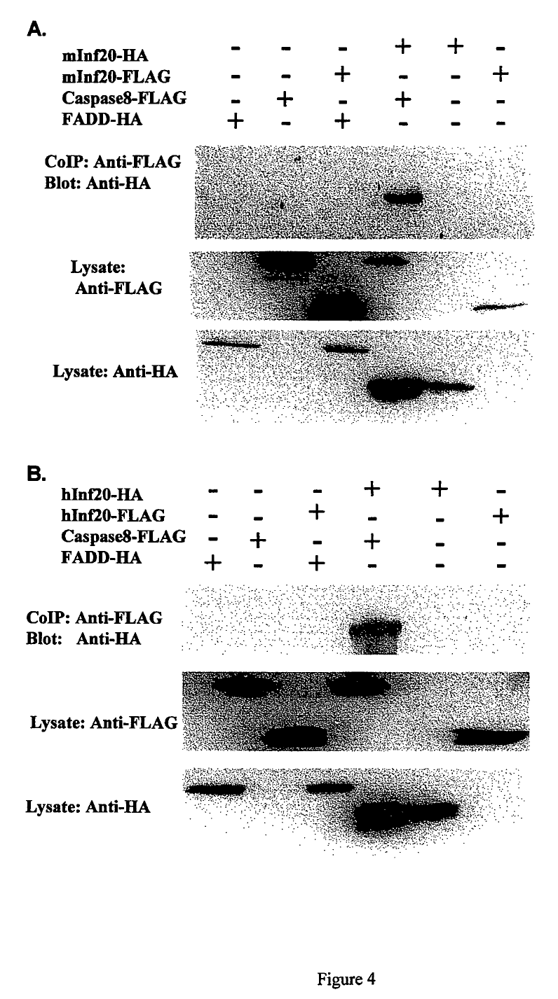

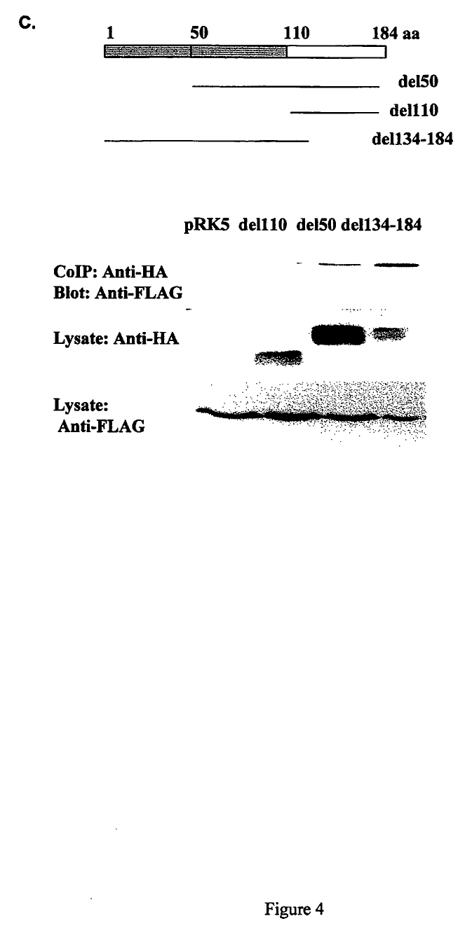

Death adapter proteins such as FADD can interact with caspases through analogous DED-DED or caspase recruitment domain (CARD)-CARD association. Since a putative DED domain was detected in Inf-20, Inf-20 was inferred to interact with FADD or caspase-8 via DED domain. To test this hypothesis, the HA-tagged Inf-20, FLAG-tagged mutant procaspase-8 (the cysteine at position 360 was replaced by serine to inactivate the enzymatic activity), FLAG-tagged Inf-20 and HA-tagged FADD were expressed in human 293 cells following transfection with corresponding expression plasmids. Co-immunoprecipitation analyses as shown in FIG. 4 revealed that both human and mouse Inf-20 specifically associated with caspase-8 but not FADD. Furthermore, an N-terminal truncated Inf-20 that does not contain DED domain was significantly compromised in its ability to bind caspase-8, whereas a C-terminal truncated Inf-20 that contains the intact DED domain retained the caspase-8-binding activity (FIG. 4C). Taken together, these results show that Inf-20 is a caspase-8-binding protein, which regulates death receptor-induced apoptosis.

### Example 4

### Inf-20 Inhibits FADD- or TNF-Induced Apoptosis

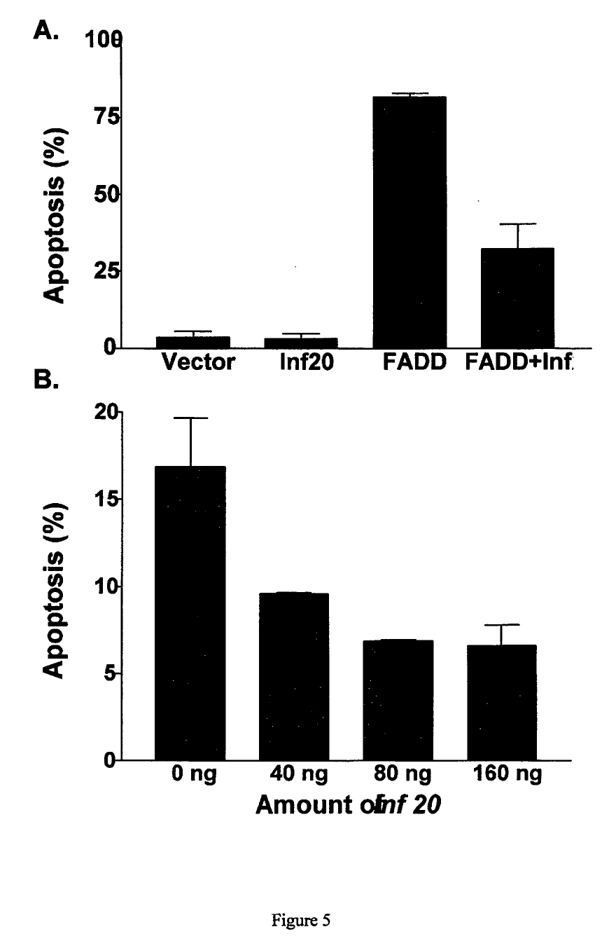

To determine the potential roles of Inf-20 in death receptor-induced apoptosis, HeLa cells were transfected with Inf-20 and FADD expression plasmids together with a LacZ expression plasmid. Apoptotic cells were identified by light microscopy based on their morphological characteristics, which include membrane blebbing, shrinkage and detachment from the plates. Only 0-galactosidase positive cells were considered to be transfected cells. FADD expression was found to induce apoptosis in more than 75% of transfected HeLa cells, which was significantly inhibited by Inf-20 expression (FIG. 5A). All cultures in FIG. 5A received the same amount of total DNA, and the rates of spontaneous cell death in vector control groups were less than 5%. The levels of FADD and Inf-20 expressions in different groups were comparable as determined by Western blot analysis.

Since Inf-20 can be activated by TNF, the roles of Inf-20 in TNF-induced apoptosis was also examined by flow cytometry. As shown in FIG. 5B, TNF induced a high degree of apoptosis in HeLa cells. This was significantly inhibited in Inf-20-transfected cells. Similar results were obtained when TNF receptor p55 was used to induce apoptosis in HeLa cells. By contrast, Inf-20 transfection had no significant effects on spontaneous cell death in the culture, which may be mediated by mitochondria-dependent intrinsic pathway. Taken together, these results show that Inf-20 is an regulator of death receptor-induced apoptotic pathway.

### Example 5

### Inf-20 is a Weak Activator of NF-κB

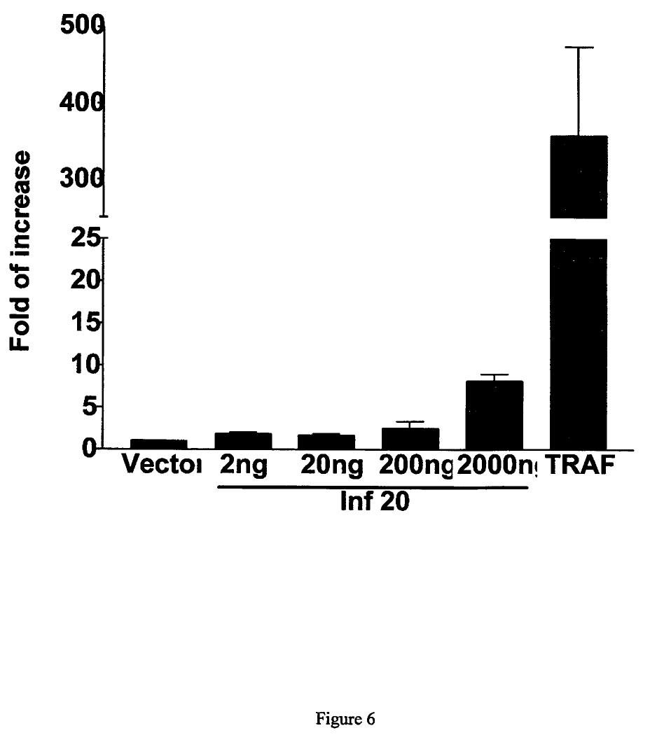

Some DED-containing proteins such as FADD and caspase-8 can activate NF-κB via a TRAF2-NIK-IKKs-dependent pathway. To determine whether NF-κB can be activated by Inf-20, HeLa cells were co-transfected with a NF-κB-luciferase reporter plasmid together with Inf-20 or TRAF-2 expression plasmid. As shown in FIG. 6, TRAF-2 expression markedly increased the NF-κB activity. By contrast, Inf-20 transfection had little effect on NF-κB activation at low doses, and only slightly increased the NF-κB activity at a very high dose, suggesting that Inf-20 may not be a strong activator of NF-κB (FIG. 6).

### Example 6

### Inf-20 Enhances Apoptosis of Primary T Cells and EL4 T Cell Line

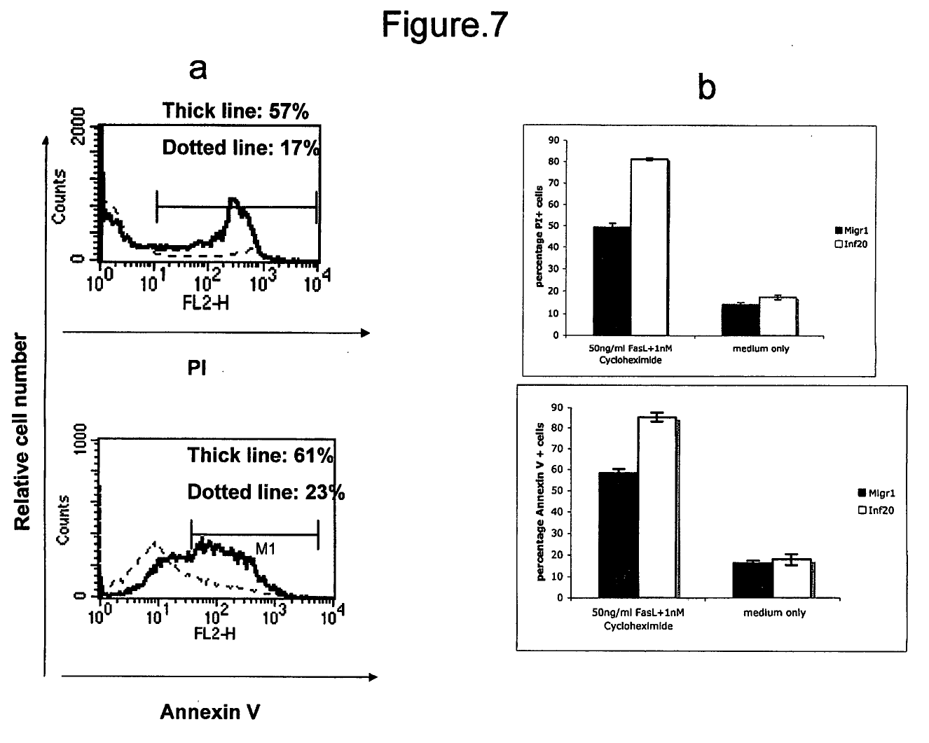

MOG-specific TCR transgenic splenocytes from Migr1-Inf-20 or Migr1 retrovirus infected bone marrow (BM) chimeric mice were stimulated for 72 hrs. Live cells were purified by Ficoll centrifugation, and re-stimulated with coated anti-CD3 (2C11, 1 ug/ml) and soluble anti-CD28 (1 ug/ml) for 16 hrs. Cells were then stained with PI or Annexin V-PE. The percentages of apoptotic cells shown were gated on NGFR+ cells (retrovirus infected only). (FIG. 7A) Dotted line, Migr1 control; thick line, Migr1-Inf20 (duplicates).

EL4 T cells infected with Migr1-Inf20 or Migr1 retrovirus were treated with FasL (50 ng/ml) and Cycloheximide (1 nM), stained with PI or Annexin V-PE. The percentages of apoptotic cells in each group of a duplicate, are shown in FIG. 7B. Shaded bar: Migr1, Open bar: Inf-20.

Results show that Inf-20 enhances Fas ligand-induced apoptosis.

### Example 7

### Inf-20 Inhibits the Proliferation of T Cells

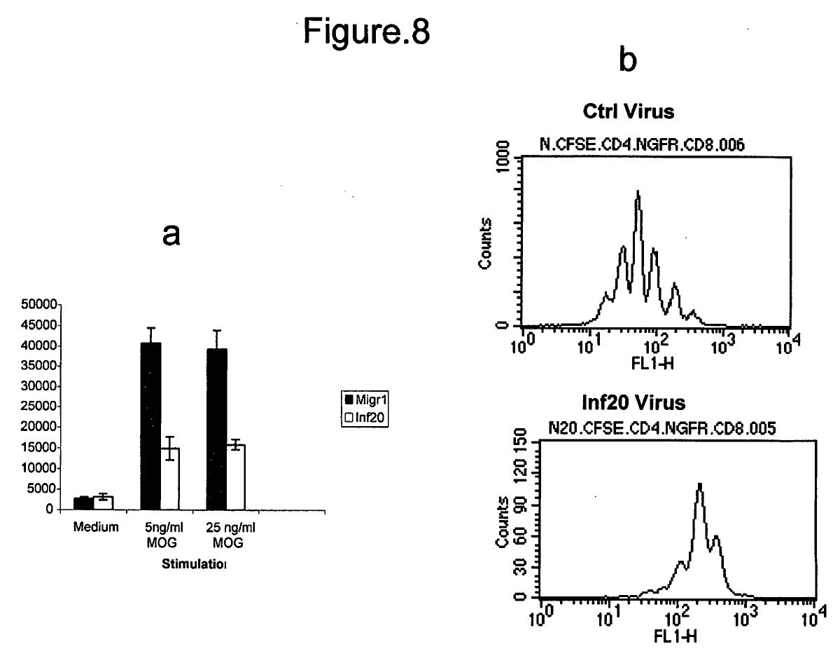

The Migr1-Inf20 or Migr1 infected bone marrow chimeric mice were immunized with MOG and sacrificed 40 days after the immunization. Splenocytes were stimulated with MOG (5 or 25 ug(ml) or medium alone for 48 hours. 3H thymidine incorporation was determined using a beta counter (FIG. 8A).

The MOG-specific TCR transgenic splenocytes of Migr1-Inf20 or Migr1 retrovirus infected BM chimeric mice were labeled with CFSE and cultured with MOG (1 ug/ml) for 72 hours. Cells were then harvested and cell division was analyzed by flow cytometry. The results were from gated NGFR+ cells (retrovirus infected only).

Results of examples 6 and 7 show, that Inf-20 promotes apoptosis of inflammatory cells, but inhibits apoptosis of the tumor cells tested.

What is claimed is:

1. An isolated Inf-20 nucleic acid molecule, encoding an apoptosis inhibition protein, wherein said Inf-20 has a nucleic acid sequence as set forth in SEQ ID NO. 1.

2. The isolated nucleic acid of claim 1, wherein said nucleic acid has a nucleic acid sequence having at least 67% similarity with the nucleic acid coding sequence of SEQ ID NO. 1.

3. The isolated nucleic acid of claim 1, wherein said nucleic acid has a nucleic acid sequence having at least 85% similarity with the nucleic acid coding sequence of SEQ ID NO. 1.

4. The isolated nucleic acid of claim 1, wherein said nucleic acid has a nucleic acid having at least 95% similarity with the nucleic acid coding sequence of SEQ ID NO. 1.

5. The isolated nucleic acid of claim 1, wherein said nucleic acid has a nucleic acid having at least 99% similarity with the nucleic acid coding sequence of SEQ ID NO. 1.

6. The isolated nucleic acid of claim 1, wherein said nucleic acid is DNA or RNA.

7. The isolated nucleic acid of any one of claims 2-5, wherein said nucleic acid is cDNA or genomic DNA.

8. The isolated nucleic acid of claim 1, wherein said nucleic acid is labeled with a detectable marker.

9. The isolated nucleic acid of claim 8, wherein the detectable marker is a radioactive, calorimetric, luminescent, fluorescent marker, or gold label.

10. An oligonucleotide of at least 15 nucleotides capable of specifically hybridizing with a sequence of said nucleic acid, which encodes the sequence of claim 1.

11. The oligonucleotide of claim 10, wherein said nucleic acid is DNA or RNA.

12. The oligonucleotide of claim 10, wherein said oligonucleotide is labeled with a detectable marker.

13. The oligonucleotide of claim 12, wherein the oligonucleotide is a radioactive, colorimetric, luminescent, fluorescent marker, or gold label.

14. A nucleic acid having a sequence complementary to the sequence of the isolated nucleic acid of claim 1.

15. An antisense molecule capable of specifically hybridizing with the isolated nucleic acid of claim 1.

16. A transgenic, nonhuman organism comprising the isolated nucleic acid of claim 1.

17. A vector comprising the isolated nucleic acid of claim 1.

18. The vector of claim 16, further comprising a promoter of RNA transcription operatively, or an expression element linked to the nucleic acid.

19. The vector of claim 18, wherein the promoter comprises a bacterial, a yeast, an insect or mammalian promoter.

20. The vector of claim 20, wherein the vector is a plasmid, cosmid, yeast artificial chromosome (YAC), BAC, adenovirus, adeno-associated virus, retovirus, P1, bacteriophage or eukaryotic viral DNA.

21. An isolated inf-20 polypeptide selected from:
a. a polypeptide comprising the amino acid sequence as set forth in SEQ ID NO:2,
b. a polypeptide comprising a naturally occurring amino acid sequence at least 72% identical to the amino acid sequence as set forth in SEQ ID NO:2, wherein said polypeptide has TNF-a or FADD induced apoptosis inhibition activity and is able to interact with caspase-8,
c. a biologically active fragment of a polypeptide having an amino acid sequence of SEQ ID NO:2, wherein said fragment has TNF-α or FADD induced apoptosis inhibition activity, or
d. a polypeptide encoded by the nucleic acid sequence as set forth in SEQ ID NO. 1.

22. An antibody of the isolated polypeptide of claim 21, wherein said antibody is a monoclonal or polyclonal antibody

23. A composition, comprising a inf-20 encoded polypeptide, represented by SEQ ID NO. 1 and a pharmaceutically acceptable carrier, excipient, flow agent, processing aid, diluent or a combination thereof

24. The composition of claim 23, wherein said carrier, excipient, lubricant, flow aid, processing aid or diluent is a gum, a starch, a sugar, a cellulosic material, an acrylate, calcium carbonate, magnesium oxide, talc, lactose monohydrate, magnesium stearate, colloidal silicone dioxide or mixtures thereof.

25. The composition of claim 23, further comprising a binder, a disintegrant, a buffer, a protease regulator, a surfactant, a solubilizing agent, a plasticizer, an emulsifier, a stabilizing agent, a viscosity increasing agent, a sweetner, a film forming agent, or any combination thereof.

26. The composition of claim 23, wherein said composition is in the form of a pellet, a tablet, a capsule, a solution, a suspension, a dispersion, an emulsion, an elixir, a gel, an ointment, a cream, or a suppository.

27. The composition of claim 23, wherein said composition is in a form suitable for oral, intravenous, intraaorterial, intramuscular, subcutaneous, parenteral, transmucosal, transdermal, or topical administration.

28. The composition of claim 23, wherein said composition is a controlled release composition.

29. The composition of claim 23, wherein said composition is an immediate release composition.

30. The composition of claim 23, wherein said composition is a liquid dosage form.

31. The composition of claim 23, wherein said composition is a solid dosage form.

32. The composition of claim 23, used for treatment, prevention, inhibition or alleviating symptoms associated with a neurological disease, wherein said neurological disease is Multiple Sclerosis (MS).

33. A method for identifying, diagnosing or predicting a candidate for developing a neurodegenerative disease comprising determining whether inf-20 is overexpressed as compared with a standard.

34. The method of claim 33, wherein overexpression of said inf-20, is by at least 25%, compared with said standard.

35. The method of claim 33, wherein said neurodegenerative disease is multiple sclerosis (MS)

36. A method of reducing symptoms of a neurodegenerative disease in a subject, comprising administering to said subject the composition of claim 23, in an amount effective to reduce symptoms of said neurodegenerative disease.

37. A method of treating a neurodegenerative disease in a subject, comprising administering to said subject the composition of claim 23, in an amount effective to treat said neurodegenerative disease.

38. A method of preventing or inhibiting neurodegenerative disease in a subject, comprising administering to said subject the composition of claim 23, in an amount effective to prevent or inhibit said neurodegenerative disease.

39. The method of any one of claims 36-38, wherein said neurodegenerative disease is multiple sclerosis (MS)

40. The method of any one of claims 36-38, wherein said composition is administered throughout the course of disease.

41. The method of any one of claims 36-38, wherein said composition is administered during symptomatic stages of disease.

42. A medium having disposed thereon an oligonucleotide-hybridized cRNA of inf-20.

43. The medium of claim 42, wherein the medium is machine readable.

44. The medium of claim 43 in the form of a microarray chip.

45. The medium of claim 42, wherein the oligonucleotide is detectably labeled.

46. A method of regulating death receptor induced apoptosis in a subject, comprising administering to said subject the composition of claim 21, in an amount effective to regulate death receptor induced Apoptosis in said subject.

47. The method of claim 44, wherein Apoptosis is induced by TNF-α, or FADD

48. The method of claim 45, wherein Apoptosis is induced by TNF-α.

49. The method of claim 45, wherein Apoptosis is induced by FADD.

50. A method of regulating death receptor induced apoptosis in a cell, comprising contacting said cell with the composition of claim 21, in an amount effective to regulate death receptor induced apoptosis in said cell.

51. The method of claim 50, wherein apoptosis is induced by TNF-α, or FADD.

52. The method of claim 50, wherein apoptosis is induced by TNF-α.

53. The method of claim 50, wherein apoptosis is induced by FADD

54. A method of promoting cell survival, comprising increasing the expression of inf-20, wherein increasing said expression of said inf-20 is by contacting said cell with an inf-20 agonist.

55. A method of screening a compound for effectiveness as an agonist of a polypeptide of claim 21, the method comprising:
a. contacting a sample comprising the polypeptide of claim 21 to a compound, and
b. determining if TNF-a or FADD induced apoptosis inhibition activity in said sample is increased in comparison to a control sample lacking said compound

56. A method of screening a compound for effectiveness as an antagonist of a polypeptide of claim 21, the method comprising:
a. contacting a sample comprising the polypeptide of claim 21 to a compound, and
b. determining if TNF-a or FADD induced apoptosis inhibition activity in said sample is decreased in comparison to a control sample lacking said compound

57. A method of blocking apoptosis in a cell comprising contacting said cell with the polypeptide of claim 21, wherein said polypeptide binds to caspase-8, therefore blocking apoptosis by directly acting on caspases.

58. A method of inhibiting TNF-α induced apoptosis in a cell, comprising increasing the expression of inf-20 in said cell thereby inhibiting TNF-a induced apoptosis in said cell.

59. A method of inhibiting FADD induced apoptosis in a cell, comprising increasing the expression of inf-20 in said cell thereby inhibiting FADD induced apoptosis in said cell.

60. The method according to claims 58 or 59, wherein increasing the expression of Inf-20 is by an Inf-20-agonist.


SEQUENCE LISTING (10 sequences)

SEQ ID NO: 1 - DNA, 552 bp, Human
atggaatctttttccagcaaatccctagcgctccaagccgaaaagaaactcttaagtaaaatggctgggagatcggtagctcacctattcattgatgaaaccagctcagaggtcctcgatgagctctatcgcgtttcgaaagagtacacgcatagtagaccacaagcacaacgggtgatcaaagacctgataaaggtagcagtcaaggttgcagtacttcaccggaatggatcttttggaccgagcgaattagcactggcgactcgttttcgtcagaagttacgacaaggtgcgatgacggctttgagtttcggggaagttgactttactttcgaagctgccgtgcttgcgggtttgcttactgaatgccgagacatactacttgagttagtcgagcatcacttaacacccaagtcacatggtcgcatcaggcatgtgtttgatcattatagtgatccaggcctgttgacagccctttacggacctgacttcacacagcacctagggaagatttgtgatggcctaaggaaattgctggacgagggaaaattg

SEQ ID NO: 2 - protein, 183 aa, Human
MESFSSKSLALQAEKKLLSKMAGRSVAHLFIDETSSEVLDELYRVSKEYTHSRPQAQRVIKDLIKVAVKVAVLHRNGSFGPSELALATRFRQKLRQGAMTALSFGEVDFTFEAAVLAGLLTECRDILLELVEHHLTPKSHGRIRHVFDHYSDPGLLTALYGPDFTQHLGKICDGLRKLLDEGK

SEQ ID NO: 3 - protein, 184 aa, Murine
MESFSSKSLALQAEKKLLSKMAGRSVAHLFIDETSSEVLDELYRVSKEYTHSRPKAQRVIKDLIKVAVKVAVLHRSGCFGPGELALATRFRQKLRQGAMTALSFGEVDFTFEAAVLAGLLVECRDILLELVEHHLTPKSHDRIRHVFDHYSDPDLLAALYGPDFTQHLGKICDGLRKLLDEGKL

SEQ ID NO: 4 - protein, 54 aa, Human
MESFSSKSLALQAEKKLLSKMAGRSVAHLFIDETSSEVLDELYRVSKEYTHSRP

SEQ ID NO: 5 - protein, 38 aa, Human
ELALATRFRQKLRQGAMTALSFGEVDFTFEAAVLAGLL

SEQ ID NO: 6 - protein, 20 aa, Human
AQRVIKDLIKVAVKVAVLHR

SEQ ID NO: 7 - protein, 19 aa, Human
ECRDILLELVEHHLTPKSH

SEQ ID NO: 8 - protein, 23 aa, Human
ALYGPDFTQHLGKICDGLRKLLD

SEQ ID NO: 9 - protein, 12 aa, Human
RIRHVFDHYSDP

SEQ ID NO: 10 - protein, 12 aa, Human
TSSEVLDELYRV